(19) United States
(12) Patent Application Publication
(10) Pub. No.: US 2006/0083680 A1
(43) Pub. Date: Apr. 20, 2006

(54) Diphenyl ether derivatives and their use for imaging serotonin transporters

(75) Inventor: Hank F. Kung, Wynnewood, PA (US)

Correspondence Address: STERNE, KESSLER, GOLDSTEIN & FOX PLLC, 1100 NEW YORK AVENUE, N.W., WASHINGTON, DC, 20005, US

(73) Assignee: The Trustees of the University of Pennsylvania, Philadelphia, PA (US)

(21) Appl. No.: 11/222,249

(22) Filed: Sep. 9, 2005

Related U.S. Application Data
(60) Provisional application No. 60/608,150, filed on Sep. 9, 2004.

(51) Int. Cl. A61K 51/00; C07C 217/54

(52) U.S. Cl. 424/1.11; 534/11; 564/389; 558/418

(57) ABSTRACT

This invention relates to diphenyl ether derivatives and their use in imaging of Serotonin Transporters (SERTS). The present invention also provides diagnostic compositions comprising the compounds of the present invention, and a pharmaceutically acceptable carrier or diluent. The invention further provides a method of imaging SERTS, comprising introducing into a patient a detectable quantity of a labeled compound of the present invention, or a pharmaceutically acceptable salt, ester, amide or prodrug thereof, allowing sufficient time for the labeled compound to associate with one or more SERTs, and detecting the labeled compound. The present invention can also be used to follow the progression of a disease associated with SERTs or a therapy that targets SERTs.

### CROSS-REFERENCE TO RELATED APPLICATIONS

This application claims benefit under 35 U.S.C. § 119(e) to U.S. Provisional Application 60/608,150, filed Sep. 9, 2004, hereby incorporated herein by reference.

### STATEMENT REGARDING FEDERALLY-SPONSORED RESEARCH AND DEVELOPMENT

Part of the work performed during the development of this invention utilized U.S. Government funds. The U.S. Government has certain rights in this invention under grant numbers EB-00369 and MH-68782 awarded by the National Institutes of Health.

### BACKGROUND OF THE INVENTION

1. Field of the Invention

This invention relates to novel radiohalogenated diphenyl ether derivatives and their use in binding and imaging of serotonin transporters (SERTs).

2. Related Art

Depression, with its related conditions, is one of the most common mental disorders in the United States. It is estimated that about five percent of the adult population experiences a depressive episode in their lifetime that requires antidepressive drug therapy. A chemical in the human brain, called serotonin, has been linked with depression and with other psychiatric disorders such as eating disorders, alcoholism, pain, anxiety and obsessive-compulsive behavior.

Abnormalities in the serotonin transporter (SERT) have been implicated in several neurologic and psychiatric disorders, such as Parkinsonian disorder, depression, suicide, schizophrenia, drug addiction and eating disorders. In order to study the above mentioned neurologic and psychiatric disorders, and the mode of action of antidepressant agents in humans, it is of great need to have high affinity and high specificity SERT radioligands for both Single Photon Emission Computerized Tomography (SPECT) and Positron Emission Tomography (PET) studies.

Serotonin (5-HT) is an essential neurotransmitter for the normal function of the central nervous system. This neurotransmission system in the brain controls various important behaviors, including sleep awake cycle, mood, temperature, appetite, etc. In addition, several commonly used anti-anxiety drugs (Frazer, A. and J. G. Hensler, Ann. NY Acad. Sci. 600:460-475 (1990); Gozlan, H. and M. Hamon, Anxiety: Neurobiol., Clinic and Ther. Persp. 232:141-150 (1993)) and antidepressants (Frazer, A., J. Clin. Psychiatry 6:9-25 (1997); Coryell, W., J. Clin. Psychiatry 1:22-27 (1998); Heninger, G. R. et al., Pharmacopsychiatry 29(1):2-11 (1996); Fuller, R. W., Prog. Drug Res. 45:167-204 (1995)) interact specifically with serotonin neurotransmission.

Pharmacological actions of the antidepressants (selective serotonin reuptake inhibitors; SSRI), such as fluoxetine (Wong, D. T. and F. P. Byrnaster, Biology 363:77-95 (1995)), paroxetine (Holliday, S. M. and G. L. Plosker, Drugs Aging 3(3)278-299 (1993)) and sertraline (Lasne, M. C. et al., Int. J. Rad. Appl. Inst.—Part A, Applied Rad Isot. 40(2):147-151 (1989)), are based on blockade of presynaptic transporters for serotonin. Thus, studies of radioligand binding to SERTs may provide valuable information of these-sites in normal and various disease states.

Several tritiated ligands including imipramine (Raisman, R. et al., Eur. J. Pharmacol. 54:307-308 (1979)), citalopram (D'Amato, R. et al., Pharmacol. Exp. Ther. 242(7):364-371 (1987)), paroxetine (Habert, E., et al., Eur. J. Pharmacol. 118(1-2):107-114 (1985)) and 6-nitroquipazine (Hashimoto, K., and T. Goromaru, Biochem. Pharmacol. 41(11):1679-1682 (1991); Hashimoto K, and T. Goromaru, Neuropharmacology 30(2):113-117 (1991)) have been used for in vitro and in vivo studies. A reduced level of SERTs labeled by these tritiated ligands has been demonstrated in post mortem brain sections of patients with depression (Perry, E. K. et al., Br. J. Psychiat. 142:188-192 (1983)), Alzheimer's and Parkinson's diseases (D'Amato, R. et al., Pharmacol Exp. Ther. 242(1):364-371 (1987)) as well as in the frontal cortex of a suicide victim (Mann, J. J., Nature Medicine 4(1):25-30 (1998)). The in vitro binding studies suggest that using in vivo imaging methods to evaluate the density of SERTs may be clinically important.

Anti-depressive drugs, such as Prozac, operate to inhibit serotonin reuptake by binding with the SERT protein, effectively blocking the binding of the protein with serotonin. Prozac is known to bind to the SERT protein, but the responses of patients can differ widely. Some patients experience greater binding than others. If a patient is not responding to Prozac treatment, there is currently no way to determine why that is the case. Frequently, the physician will simply administer greater doses of the drug, a practice which will not necessarily lead to better results and which can enhance undesirable side effects.

SPECT and PET are well known nuclear imaging systems in medicine. Generally, in nuclear imaging, a radioactive isotope is injected to, inhaled by or ingested by a patient. The isotope, provided as a radioactive-labeled pharmaceutical (radio-pharmaceutical) is chosen based on bio-kinetic properties that cause preferential uptake by different tissues. The gamma photons emitted by the radio-pharmaceutical are detected by radiation detectors outside the body, giving its spatial and uptake distribution within the body, with little trauma to the patient.

SPECT imaging is based on the detection of individual gamma rays emitted from the body, while PET imaging is based on the detection of gamma-ray pairs that are emitted in coincidence, in opposite directions, due to electron-positron annihilations. In both cases, data from the emitted photons is used to produce spatial images of the “place of birth” of a detected photon and a measure of its energy.

Development of selective tracers for PET and SPECT have made it possible to study in vivo neuroreceptors or site-specific bindings non-invasively in the human brain. However, development of PET or SPECT tracers specifically for in vivo imaging of SERT has only met with limited success. A promising radioligand described to date is [¹¹C](+)McN5652 for PET imaging (Szabo, Z. et al., Synapse 20(1):37-43 (1995); Szabo, Z. et al., J. Nucl. Med. 37(5):125 (1996); Szabo, Z. Behav. Brain Res. 73(1):221-224 (1995); Szabo, Z. et al., J. Cerebral Blood Flow & Metabol. 15(5):798-805 (1995); Suchiro, M. et al., J. Nucl. Med. 34(1):120-127 (1993); Suchiro, M. et al., Nucl. Med. Biol. 22(4):543-545 (1995)). Specific binding of [¹¹C](+)McN5652 correlates well with the known density of SERT sites in the human brain (Szabo, Z. et al., Synapse 20(1):37-43 (1995)).

Several radioligands have been developed for PET studies of SERTs. These include fluorine-18 labeled paroxetine, fluoxetine and carbon-11 labeled cyanoimipramine, citalopram, sertraline and fluoxetine. All of these radioligands were found not to be the ideal agents for PET studies of SERTs due to their low specific-to-nonspecific binding ratios in vivo. For the last decade, [¹¹C](+)McN-5652 has been the most promising PET agent for studying SERTs in humans. However, this agent has high nonspecific binding and has only moderate signal contrast in human PET studies. Additionally, its pharmacokinetics profile is not optimal due to the short half-life of carbon-11.

While the SSRI drugs have helped many patients, a significant segment of patients does not respond to the treatment. There is a compelling need to find a simple method to measure the drug occupancy (or the lack thereof) of the target sites in the brain of non-responders, and agents useful for such a method. Noninvasive imaging methods are important for studying binding sites of psychoactive drugs and monitoring the effectiveness of such drug treatment in the living human brain.

### SUMMARY OF THE INVENTION

The present invention is directed to compounds of Formulae I-III:

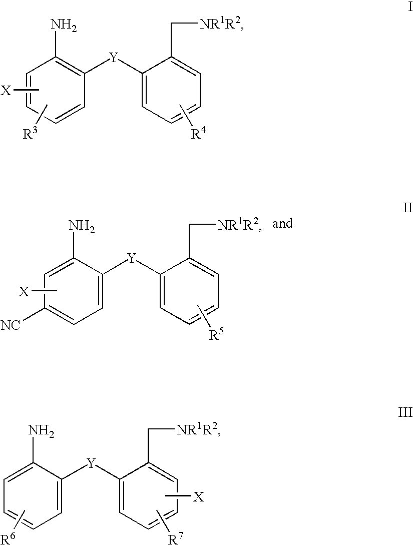

wherein X, Y, R¹, R², R³, R⁴, R⁵, R⁶ and R⁷ are as defined below.

The present invention is also directed: to radiohalogenated compositions comprising a compound of Formulae I, II or III and a pharmaceutically acceptable carrier or diluent.

The invention further provides a method of imaging Serotonin Transporters (SERTs) utilizing compounds of Formulae I, II or III.

### BRIEF DESCRIPTION OF THE FIGURES

FIGS. 1A and 1B depict the comparison of uptake in hypothalamus (1A) and specific uptake ratio (1B) of compounds including, ADAM (3) at different time points after an i.v. injection into rats. Post i.v. injection into rats: The specific uptake in hypothalamus is expressed as % dose/g tissue (graph A); while the specific uptake ratio is expressed as % dose/g ratio of (hypothalamus-cerebellum)/cerebellum (graph B).

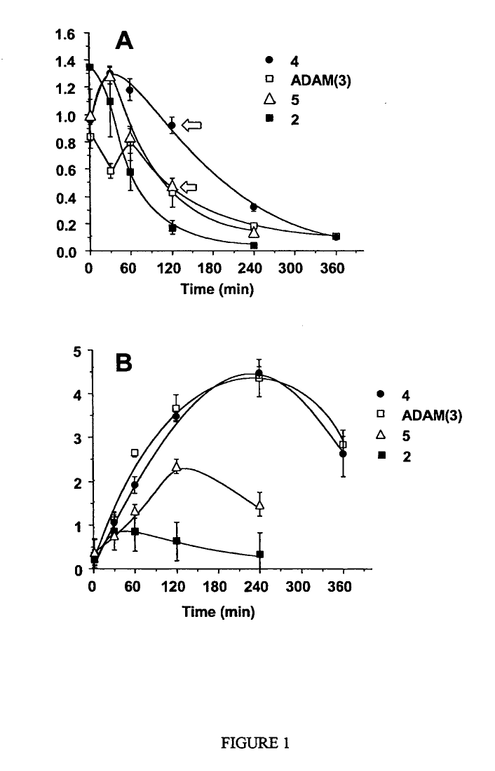

FIGS. 2A and 2B depict ex vivo autoradiographic localization of [¹²⁵I]4 binding sites in rats after 120 min post-injection. (A) control rat—High levels of radioactivity were observed in areas containing high densities of serotonin transporter sites. The coronal sections corresponding to the stereotaxic atlas. Abbreviations: CA1, CA3: CA1, CA3 Ammon's horn; CP: caudate putamen; Cx: cortex; DR: dorsal raphe nucleus; GP: globus pallidus; IP: interpedunclar nucleus; MnR: median raphe nucleus; LH: Lateral hypothalamic area; LThN:laterodorsal thalamic nucleus; OT: olfactory tubercle; PH: periventricular hypothalamic nucleus; SC: superior colliculus; SN: substantia nigra; ThN: thalamus nucleus; VLG: Ventricular lateral geniculate nucleus. (B) a rat pretreated with a dose of (+)McN5652 (2 mg/Kg body weight) at 5 min prior to the i.v. injection of [¹²⁵I]4.

FIG. 3 depicts in vivo blockade of [¹²⁵I]4 after pretreatment with various compounds on specific binding in rat brain regions. In vivo blockade of [¹²⁵I]4 after pretreatment with various compounds on specific binding in rat brain regions. The specific binding is expressed as the ratios of (region-cerebellum)/cerebellum, [(Region-CB)/CB], on a % dose/g basis. Rats were pretreated with various drugs with a dose of drug (2 mg/kg, i.v. at 5 min prior to tracer administration). Two hours after the tracer injection, specific binding in each brain region was compared between saline-pretreated (control) and drug-pretreated rats. The specific binding is expressed as the ratios of (region-cerebellum)/cerebellum, [(Region-CB)/CB], on a % dose/g basis. Values are presented as the average±SD of three rats in each point. (+)McN5652, escitalopram and IDAM—serotonin transporter ligand; methylphenidate-dopamine transporter ligand; nisoxetine-norepinephrine transporter ligand; raclopride-dopamine D₂/D₃ receptor antagonist; ketanserin -5-HT₂ receptor antagonist.

### DETAILED DESCRIPTION OF THE INVENTION

A first aspect of the invention is directed to compounds of Formula I: 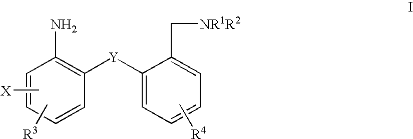
or a pharmaceutically acceptable salt thereof, wherein:

Y is O or CH₂,

one of R¹ and R² is hydrogen or C₁₋₄ alkyl, the other of R¹ or R² is C₁₋₄ alkyl,

R³ is selected from the group consisting of hydrogen and C₁₋₄ alkyl,

R⁴ is selected from the group consisting of hydrogen, C₁₋₄ alkyl and halo(C₁₋₄)alkyl, and

X is ¹³¹I, ¹²³I, ¹²⁵I, ¹⁸F, ⁷⁶Br, ⁷⁷Br ¹⁸Fluoro(C₁₋₄)alkyl; or CF₃CF₂(CH₂)ₙ—, wherein at least one F is ¹⁸F; and n is zero or one.

Preferred values of R¹ include all of those values listed above. It is more preferred that one of R¹ and R² is hydrogen or C₁₋₄ alkyl and the other of R¹ and R² is C₁₋₄ alkyl. In these embodiments, it is more preferred that R¹ is hydrogen, methyl or ethyl and R²⁻ is independently selected from the group consisting of methyl or ethyl. In most preferred embodiments, R¹ and R² are both methyl.

Preferred values of R³ and R⁴ include all of those values listed above. Each of R³ and R⁴ is selected independently from the other. In most preferred embodiments, R³ is hydrogen. In most preferred embodiments, R⁴ is hydrogen.

Useful values of X include those values listed above. In more preferred embodiments, X is ¹³¹I, ¹²³I, ¹²⁵I, ¹⁸F or ¹⁸Fluoro(C₁₋₄)alkyl. In most preferred embodiments, X is ¹²³I or ¹²⁵I.

Useful values of Y include —CH₂— and O. In all embodiments described herein, Y is most preferably O.

One embodiment is directed to compounds of Formula I as described above; provided that if X is 4-¹²³I or 4-¹²⁵I, R¹ and R² are both methyl, and R⁴ is hydrogen, then R³ is other than hydrogen.

Most preferred embodiments include compounds having the following structures:

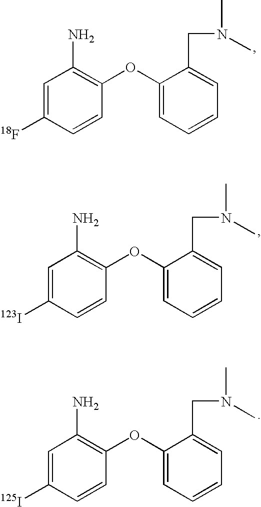

Other preferred compounds of Formula I include the following structures, wherein at least one atom represented as “F” is ¹⁸F:

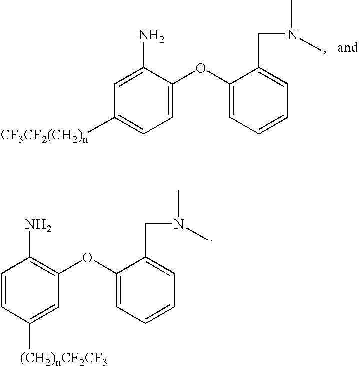

The present invention is also directed to compounds of Formula II: 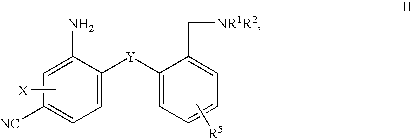
or a pharmaceutically acceptable salt thereof; wherein,

Y is O or CH₂,

one of R¹ and R² is hydrogen or CIA alkyl, the other of R¹ or R² is C₁₋₄ alkyl,

R⁵ is selected from the group consisting of hydrogen, C₁₋₄ alkyl and halo(C₁₋₄)alkyl, and

X is ¹³¹I, ¹²³I, ¹²⁵I, ¹⁸F, ⁷⁶Br, ⁷⁷Br, ¹⁸Fluoro(C₁₋₄)alkyl; or CF₃CF₂(CH₂)ₙ—, wherein at least one F is ¹⁸F, and n is zero or one.

Preferred values of Y include —CH₂— and O. In all embodiments described herein, Y is most preferably O.

Preferred values of R¹ include all of those values listed above. It is more preferred that one of R¹ and R² is hydrogen or C₁₋₄ alkyl and the other of R¹ and R² is C₁₋₄ alkyl. In these embodiments, it is more preferred that R¹ is hydrogen, methyl or ethyl and R² is independently selected from the group consisting of methyl or ethyl. In most preferred embodiments, R¹ and R² are both methyl.

Preferred values of R⁵ include all those listed above. In all embodiments, R¹ is most preferably hydrogen.

Useful values of X are described above. In preferred embodiments, X is ¹³¹I, ¹²³I, ¹²⁵I, ¹⁸F, ¹⁸Fluoro(C₁₋₄)alkyl; or CF₃CF₂(CH₂)ₙ—, wherein at least one F is ¹⁸F, and n is zero or one. Most preferably X is ¹⁸F, or CF₃CF₂(CH₂)ₙ—. When X is CF₃CF₂(CH₂)ₙ—, at least one F atom is ¹⁸F. Preferred compounds include those compounds where n is zero or one.

Most preferred embodiments include compounds having the following structures:

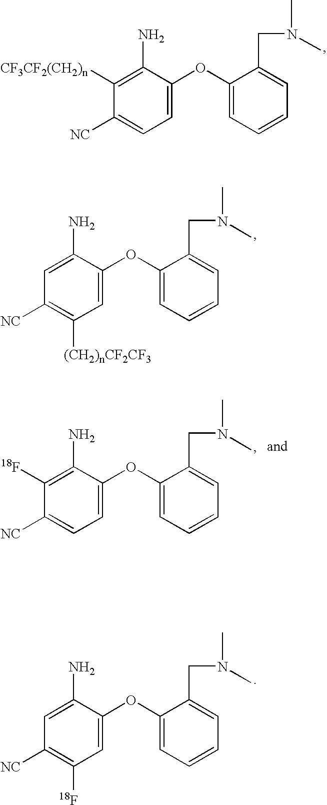

The present invention is also directed to compounds of Formula III: 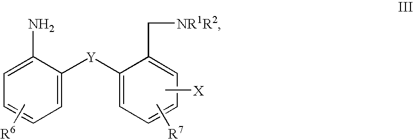
or a pharmaceutically acceptable salt thereof; wherein,

Y is O or CH₂,

one of R¹ and R² is hydrogen or C₁₋₄ alkyl, the other of R¹ or R² is C₁₋₄ alkyl,

R⁶ is selected from the group consisting of hydrogen, halo(C₁₋₄)alkyl and C₁₋₄ alkyl,

R⁷ is selected from the group consisting of hydrogen, C₁₋₄ alkyl, cyano, ¹⁸F and CF₃CF₂(CH₂)ₙ—, wherein at least one F is ¹⁸F, and n is zero or one, and

X is hydrogen, cyano, ¹³¹I, ¹²³I, ¹²⁵I, ¹⁸F, ⁷⁶Br, ⁷⁷Br, ¹⁸Fluoro(C₁₋₄)alkyl; or CF₃CF₂(CH₂)ₙ—, wherein at least one F is ¹⁸F, and n is zero or one.

Preferred values of Y include —CH₂— and O. In all embodiments described herein, Y is most preferably O.

Preferred values of R¹ include all of those values listed above. It is more preferred that one of R¹ and R² is hydrogen or C₁₋₄ alkyl and the other of R¹ and R² is C₁₋₄ alkyl. In these embodiments, it is more preferred that R¹ is hydrogen, methyl or ethyl and R² is independently selected from the group consisting of methyl or ethyl. In most preferred embodiments, R¹ and R² are both methyl.

Preferred values of R⁶ include those listed above. In all embodiments, R⁶ is most preferably hydrogen.

Useful values of R⁷ include those listed above. In preferred embodiments, R⁷ is selected from the group consisting of cyano, ¹⁸F and CF₃CF₂(CH₂)ₙ—, wherein at least one F is ¹⁸F, and n is zero or one.

Useful values of X are described above. In preferred embodiments, X is cyano, ¹³¹I, ¹²³I, ¹²⁵I, ¹⁸F, ¹⁸Fluoro(C₁₋₄)alkyl; or CF₃CF₂(CH₂)ₙ—, wherein at least one F is ¹⁸F, and n is zero or one. Most preferably X is ¹⁸F, or CF₃CF₂(CH₂)ₙ—. When X is CF₃CF₂(CH₂)ₙ—, at least one F atom is ¹⁸F. Preferred compounds include those compounds where n is zero or one.

In more preferred embodiments, R⁷ and X are different from each other. An example of this embodiment includes the following structure, where R⁷ is in the −5 position:

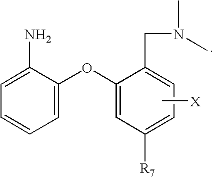

In this embodiment, preferred compounds include those compounds where R⁷ is hydrogen and X is cyano, ¹⁸F, or CF₃CF₂(CH₂)ₙ—, wherein at least one F is ¹⁸F, and n is zero or one. In another preferred embodiment, X is hydrogen and R⁷ is cyano, ¹⁸F, or CF₃CF₂(CH₂)ₙ—, wherein at least one F is ¹⁸F, and n is zero or one. In another preferred embodiment, R⁷ is hydrogen, ¹⁸F, or CF₃CF₂(CH₂)ₙ—, wherein at least one F is ¹⁸F, and n is zero or one, and X is cyano. More preferred compounds in this embodiment have the following structures:

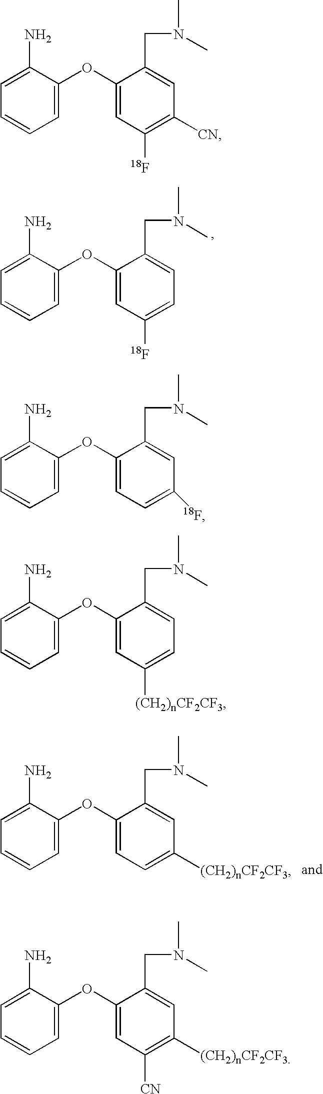

Other preferred compounds of this embodiment include those compounds where R⁷ is cyano, and X is ¹⁸F, or CF₃CF₂(CH₂)ₙ—, wherein at least one F is ¹⁸F, and n is zero or one. Examples of these compounds include compounds of the following structures:

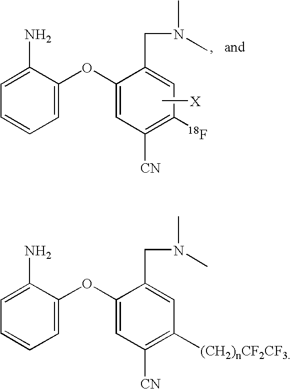

Another aspect of the present invention is directed to a pharmaceutical composition comprising a compound of the present invention, and a pharmaceutically acceptable excipient or diluent.

In another aspect, the present invention is directed to a diagnostic composition for imaging serotonin transporters, comprising a compound of the present invention, and a pharmaceutically acceptable excipient or diluent.

In another aspect, the present invention is directed to a method of imaging SERTs comprising introducing into a patient a detectable quantity of a labeled compound of Formulae I, II or III or a pharmaceutically acceptable salt, ester, amide or prodrug thereof, allowing sufficient time for the labeled compound to be associated with SERTs, and detecting the labeled compound associated with one or more SERTs.

In another aspect, the present invention is directed to methods of diagnosing depression and following the progress of a patient thought to suffer from depression. The SERT imaging agents described herein can provide quantitative information on SERT binding sites in the brain. When patients receive SSRI drug treatment that target SERT sites, these imaging agents can measure the occupancy of the SERT binding sites. This can provide information on the action of the drug, or lack thereof, in the targeted brain area. The method of diagnosing depression and following the progress of a patient who is being evaluated for depression comprises comprising introducing into a patient a detectable quantity of a labeled compound of Formulae I, II or III or a pharmaceutically acceptable salt, ester, amide or prodrug thereof, allowing sufficient time for the labeled compound to be associated with SERTs, detecting the labeled compound associated with one or more SERTs, and determining an amount of SERTs affected by the anti-depressive therapy. This method is equally useful for following the progress of any therapy which targets SERTs.

In yet another aspect, the present invention is directed to a method of preparing the compounds of the present invention through the syntheses outlined below.

When any variable occurs more than one time in any constituent or in Formula I its definition on each occurrence is independent of its definition at every other occurrence. Also combinations of substituents and/or variables are permissible only if such combinations result in stable compounds.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “halo” employed herein by itself or as part of another group refers to chlorine, bromine, fluorine or iodine.

The term “halo(C₁₋₄)alkyl” as employed herein refers to any C₁₋₄ alkyl group substituted by one or more chlorine, bromine, fluorine or iodine with fluorine being most preferred.

As used herein, the term “¹⁸Fluoro(C₁₋₄)alkyl” refers to a halo(C₁₋₄)alkyl as described above that is substituted with a single ¹⁸F atom in any available position on the alkyl. Most preferably, the ¹⁸F atom is located on the terminal carbon distal to the attachment of the alkyl to a molecule of Formulae I, II or III.

Imaging SERTs, the target-sites for commonly used antidepressants, such as fluoxetine, paroxetine, citalopram and sertraline etc, is an emerging research tool potentially useful to cast light on the mechanisms of drug action as well as to monitor the treatment of depressed patients. We have prepared two new derivatives of 3,2-(2-(dimethylanainomethyl)phenoxy)-5-iodophenylamine (4) and 2-(2-(Dimethylaminomethyl)benzyl)-5-(tributyltin)phenylamine (5), containing either an oxygen or a carbon between the biphenyl ring instead of a sulfur. These two compounds, 4 and 5, exhibited in vitro binding affinities towards SERTs (Kᵢ=0.37 and 48.6 nM, respectively). Both [¹²⁵I]4 and [¹²⁵I]5 displayed excellent brain uptakes in rats (1.77±0.07% dose and 1.77±0.049% dose at 2 min post-injection, respectively). Importantly, compared to iodinated compounds, such as [¹²⁵I]ADAM (3) and [¹²⁵I]5, [¹²⁵I]4 consistently displayed the highest uptake in hypothalamus (between 60-240 min post i.v. injection), a region populated with the highest density of SERTs. Nevertheless, with a slower kinetic washout of the tracer from the brain regions, the specific uptake of [¹²⁵I]4 in the hypothalamus resulted in a target to non-target ratio ([hypothalamus-cerebelhlum]/erebellum) of 4.3 at 240 min post i.v. injection. The specific hypothalamic uptake were significantly blocked by SERT agents, i.e. (+)McN5652 and citalopram. Autoradiography of rat brain sections (ex vivo at 3 h post i.v. injection) of [¹²⁵I]4 showed an excellent regional distribution pattern consistent with known SERT localization. These data clearly suggest that the new ligand, [¹²³I]4, may be useful as a molecular imaging agent for SERT binding sites in the brain by SPECT.

The following schemes depict a synthetic route for preparing several of the compounds of the present invention. Synthesis of 4, an O-bridged derivative, is outlined in Scheme 1(A). 2-Dimethylaminomethylphenol (6) and 2,5-dibromonitrobenzene (7) were coupled to afford compound 8. The nitro group of 8 was reduced to corresponding amine 9 by SnCl₂. Compound 9, a bromo derivative of target compound, 4, was converted to a tributyltin intermediate 10 by a palladium (0) catalyzed coupling reaction with bis(tributyltin). The tin compound 10 was treated with iodine in chloroform to produce the desired compound, 4.

Synthesis of 5, a C-bridged derivative, is outlined in Scheme 1(B). 2-Bromo-N,N′-dimethylbenzylamine (11) was lithiated by n-BuLi and reacted with either 4-bromo-2-nitrobenzaldehyde See Jung, M. E., et al., Heterocycles, 39, 767-778, (1994). (12a), or 4-iodo-2-nitrobenzaldehyde (12b), leading to the carbon-bridged alcohol, 13a and 13b. The alcohols were readily reduced by borane to give the bromo derivative, 14 and the desired “cold” compound, 5. The bromo derivative, 14, was converted to the tributyltin derivative, 15.

To prepare the radioiodinated ligands, compound 10 was treated with radioactive [¹²³I]/[¹²⁵I] sodium iodide in an oxidative condition (H₂O₂) to produce the labeled compound [¹²³I]/[¹²⁵I]4 in excellent yields (>90%). See Scheme 2. The radioactive material, [¹²⁵I]4, is stable in a refrigerator for at least three weeks and the corresponding [¹²³I]4 was stable for 24 h after labeling. Tin compound 15 was treated with radioactive [¹²⁵I] sodium iodide in an oxidative condition (H₂O₂) to produce the labeled compound [¹²⁵I]5 in excellent yields (>90%). The radioactive material [¹²⁵I]5 is stable in a refrigerator at least for three weeks.

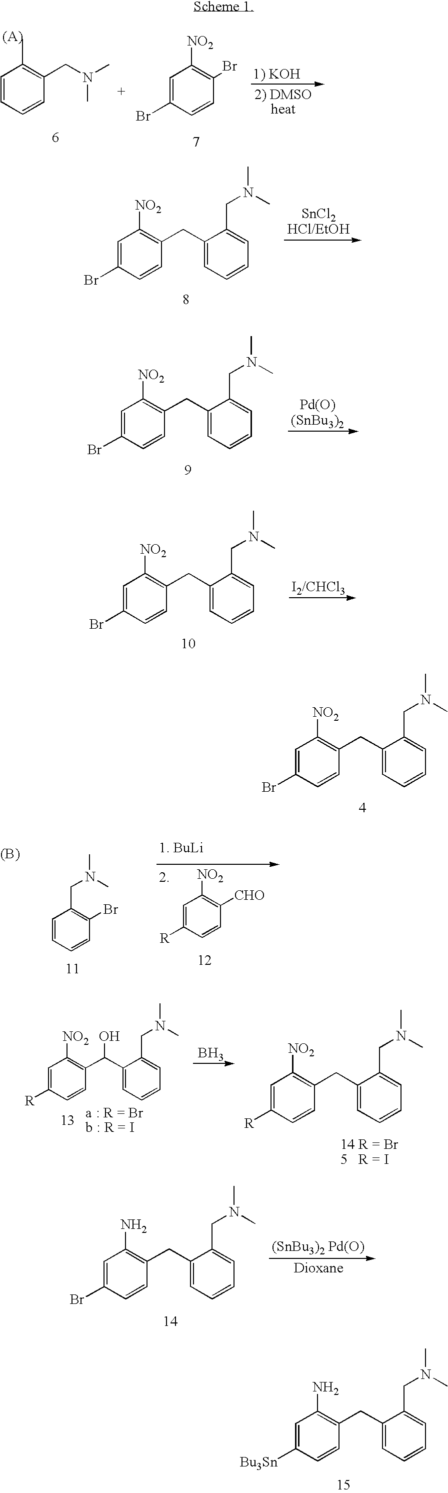

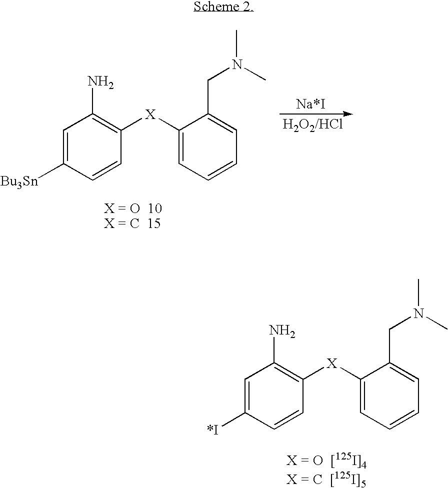

Schemes 3 through 8 depict synthetic routes for preparing compounds of Formula II.

In Scheme 3, nitration of the aromatic ring in step 1 proceeded through the method of Kudo, et al., Chem. Pharm. Bull., 44: 9, 1996, 1663-1668. Reduction of the nitro group using aq. NH₄Cl was accomplished by the method of Moody, et al., Syn. Lett., 9, 1998, 1028. Fluorination was performed by the method of Sasson, et al., JCS Chem. Commun., 3, 1996, 297-298.

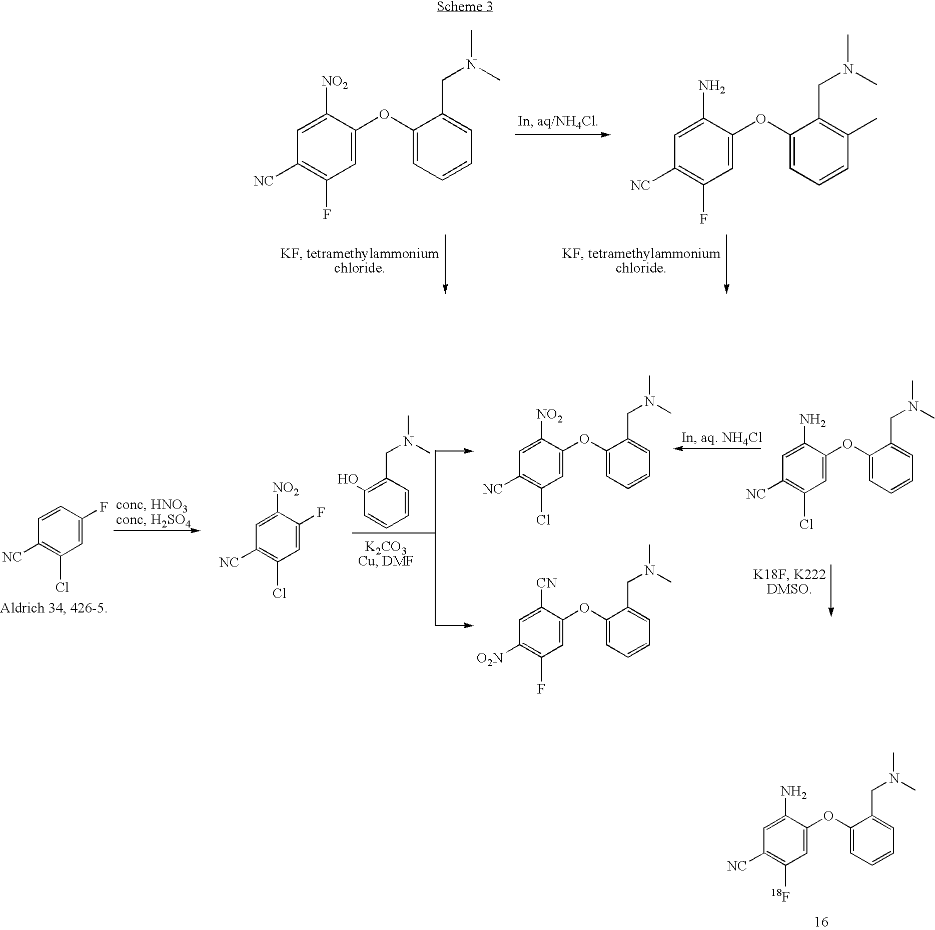

In Scheme 4, Iodination was performed using the method of Anilkumar, et al., Tet. Lett., 43, 2002, 2731. Fluorinated ethylene was added to the ring via the method of Bolbier, et al., Appl. Rad. Isotopes, 54: 2001, 73.

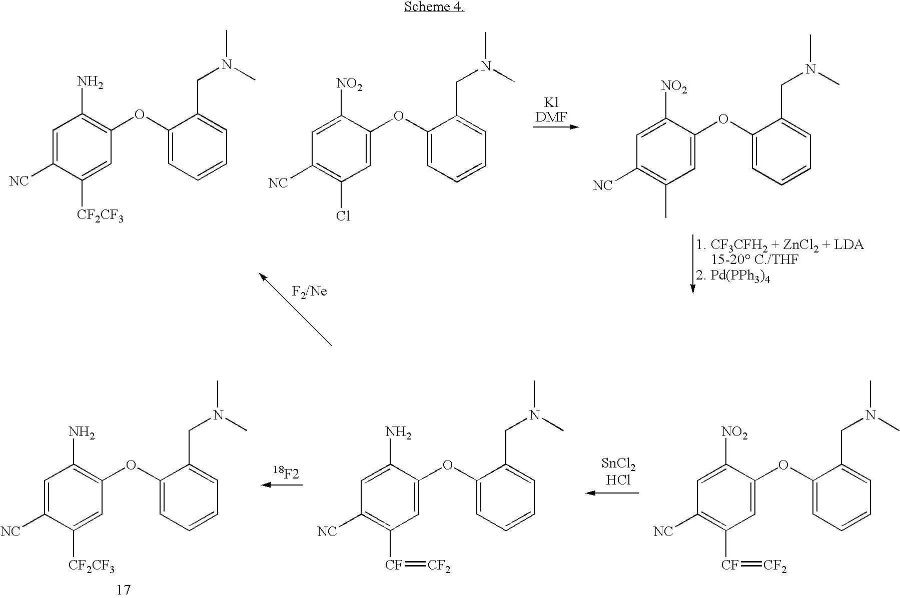

In Scheme 5, replacement of I with fluorinated 2-propylene was accomplished using the method of Burkhardt, et al., JOC, 50, 1985, 416.

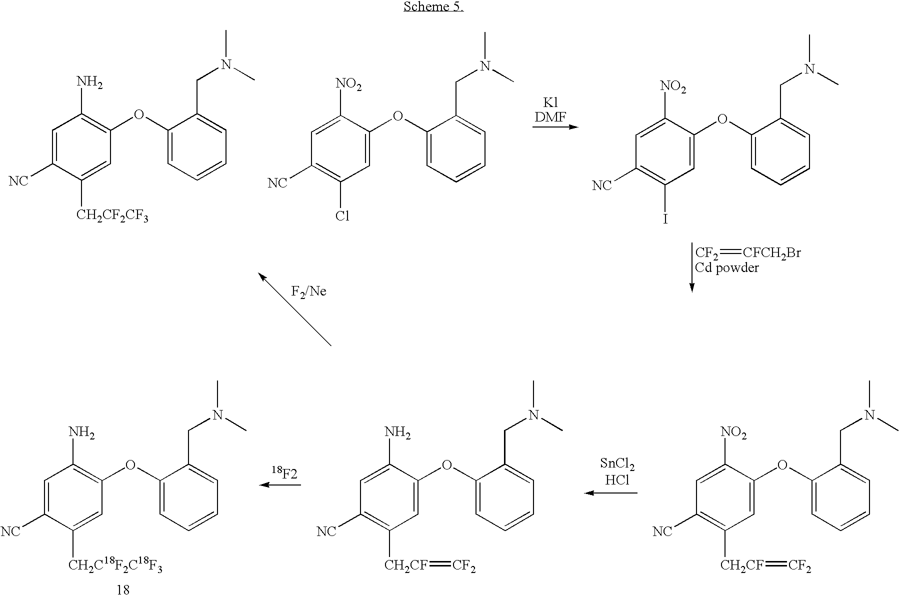

In Scheme 6, nitration was accomplished using the method outlined in EP 282944 (1988) and Helv. Chim. Acta, 40, 1957, 1197.

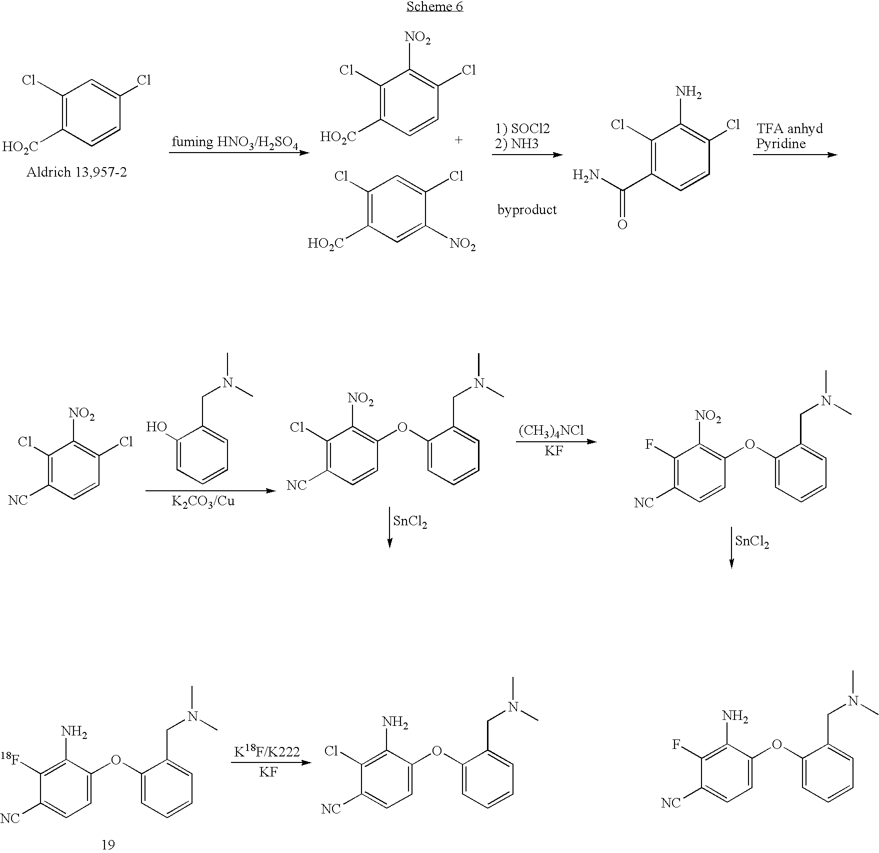

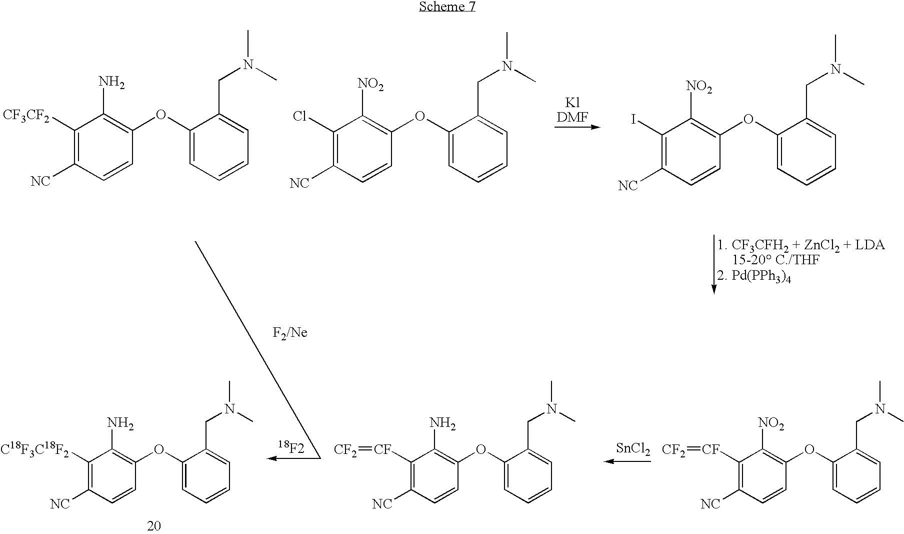

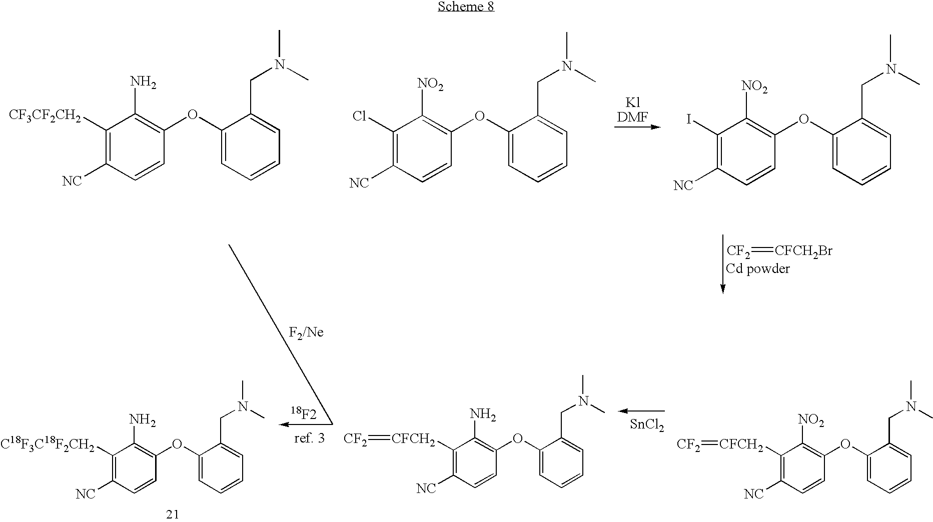

Schemes 9 through 14 depict synthetic routes for preparing compounds of Formula III.

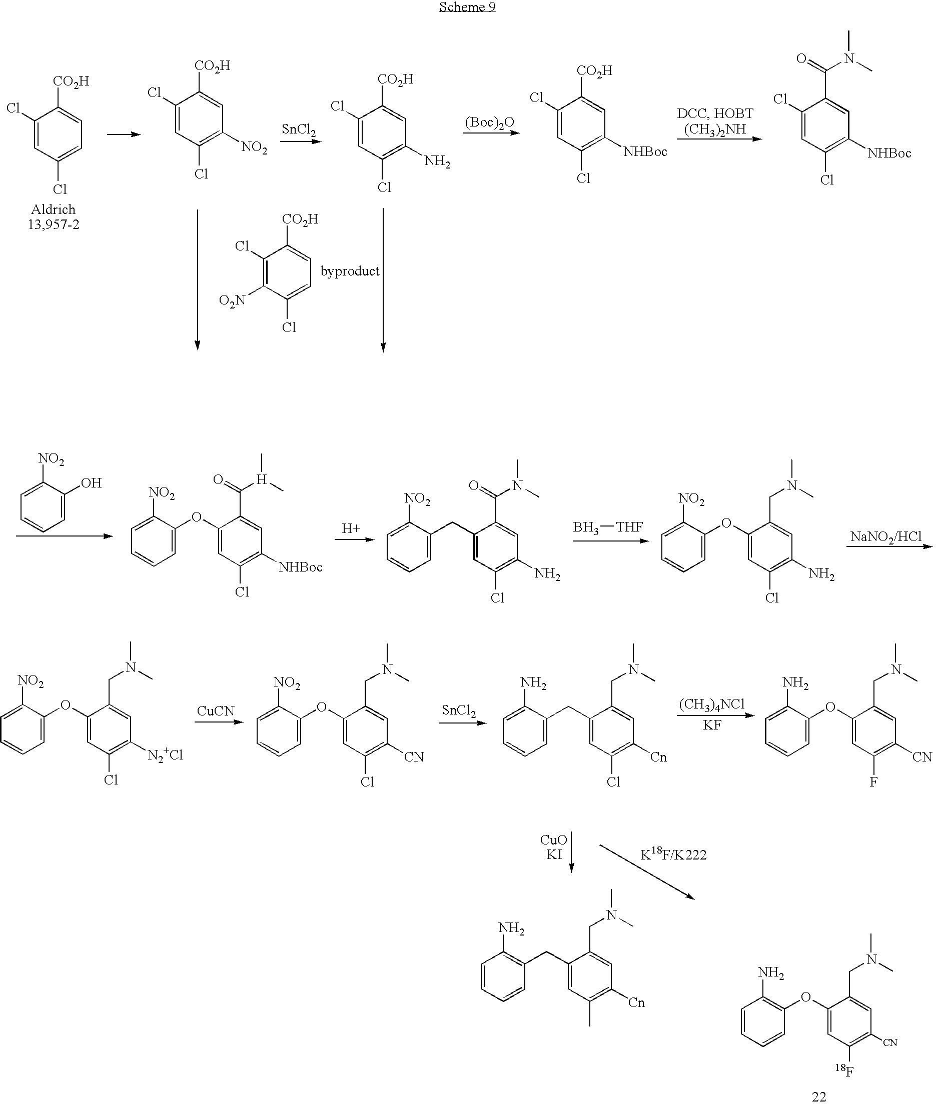

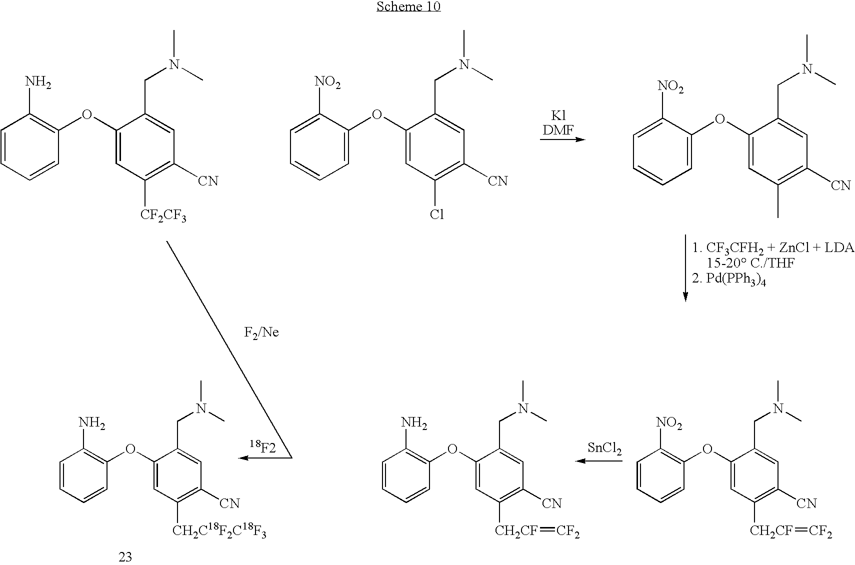

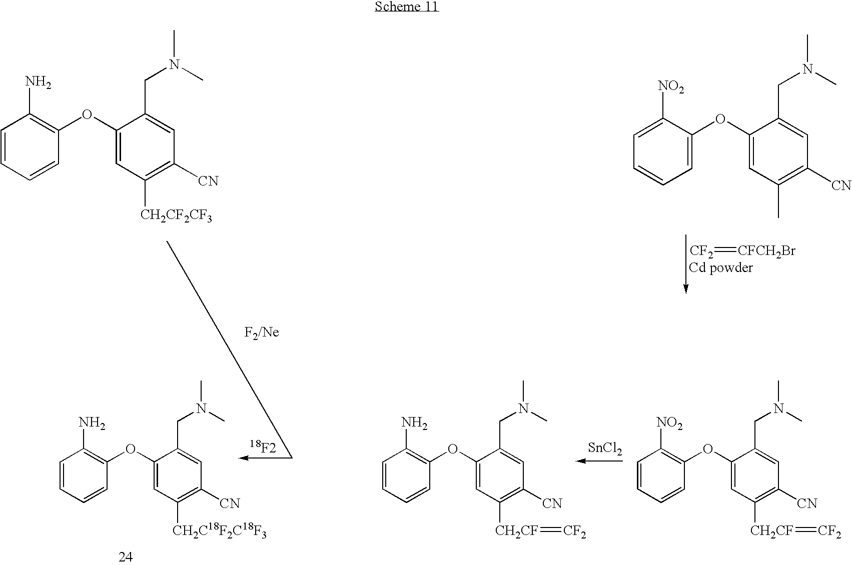

In Scheme 12, nitration was accomplished using the method described in JACS, 79, 1901, 1130. Carboxylation was performed using the method described in JACS, 75, 1953, 4675.

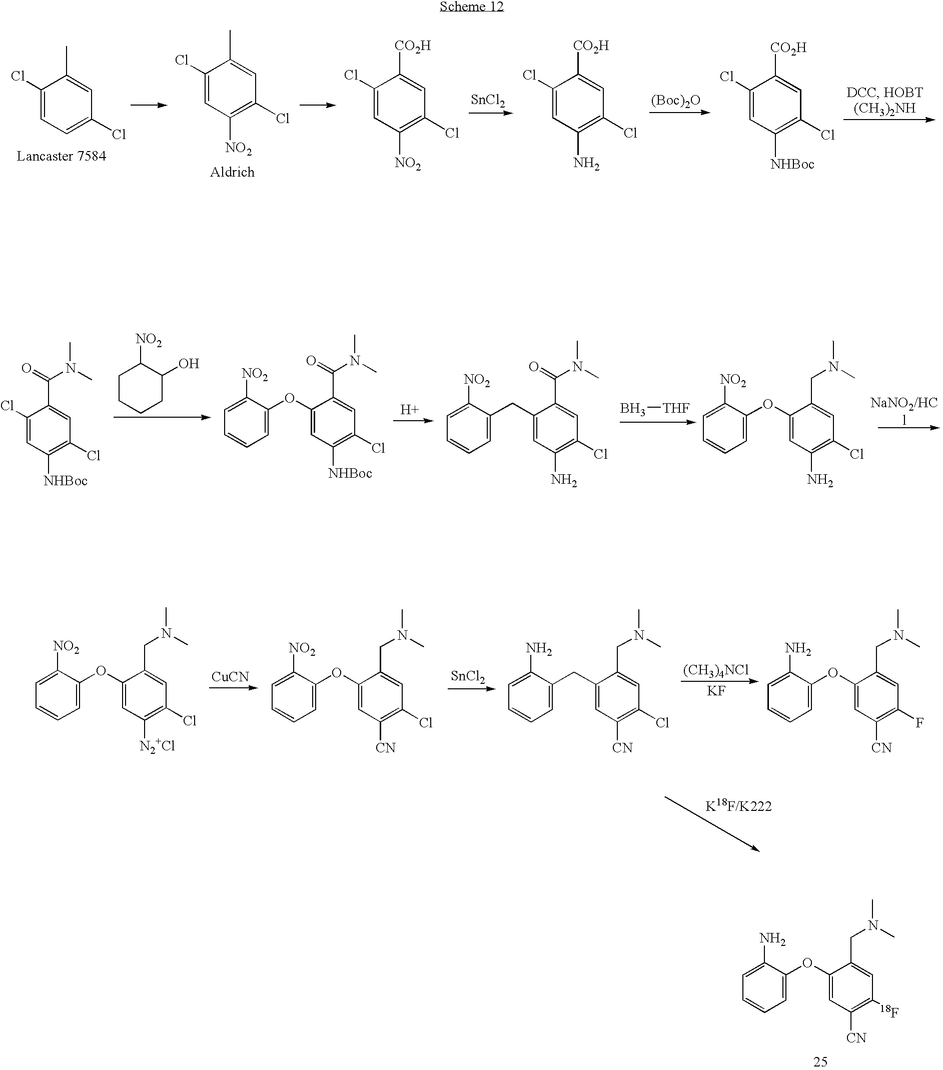

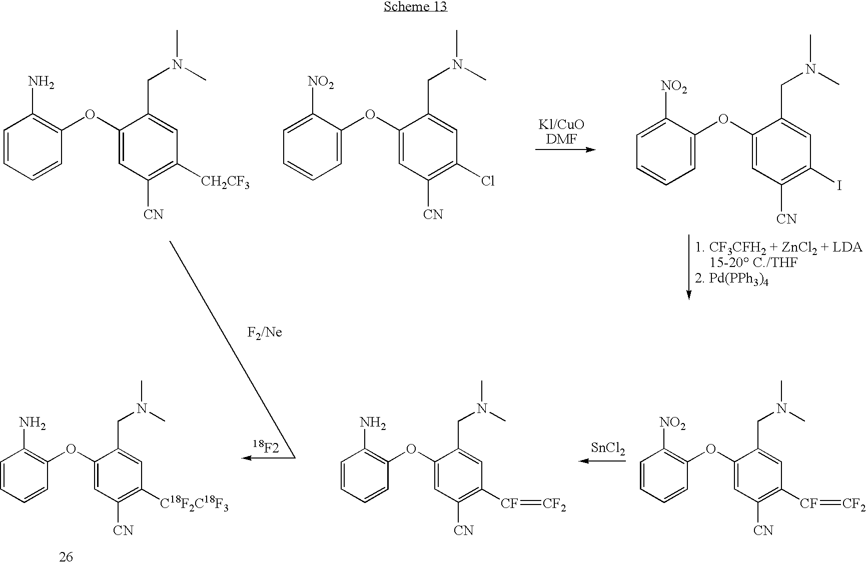

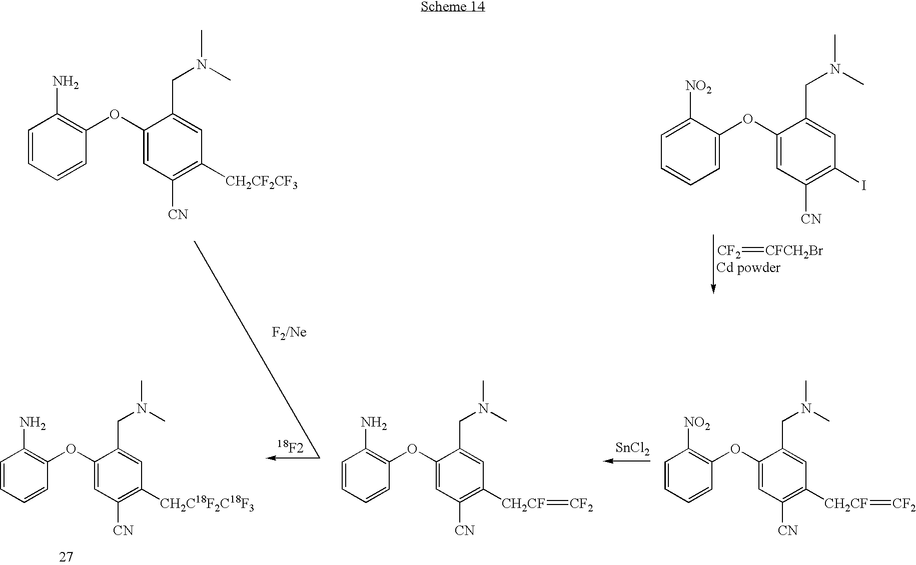

Schemes 15 through 20 depict synthetic routes for preparing compounds of Formula I.

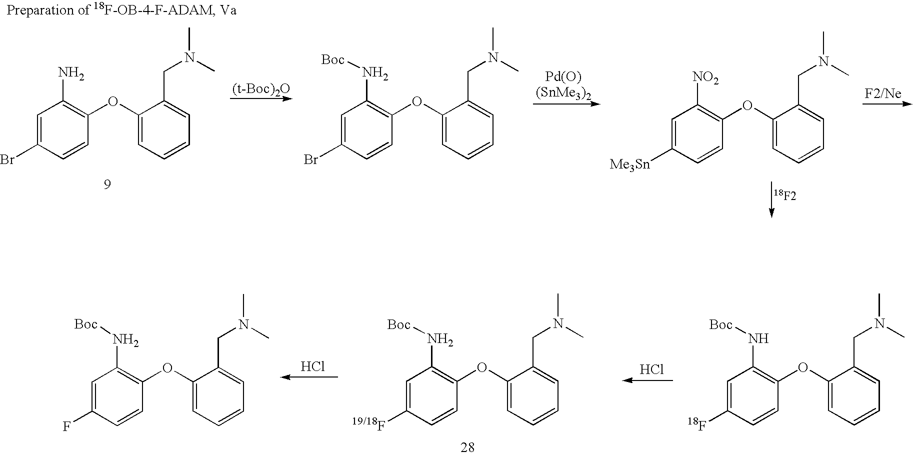

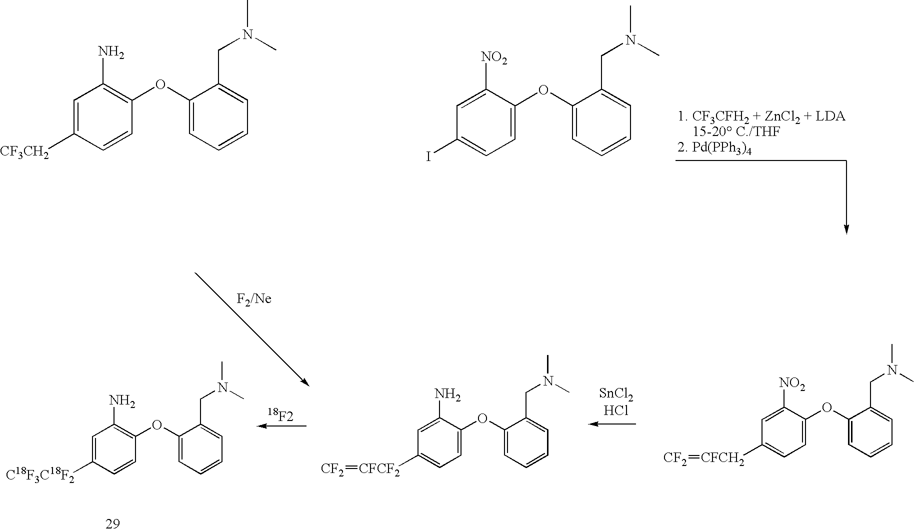

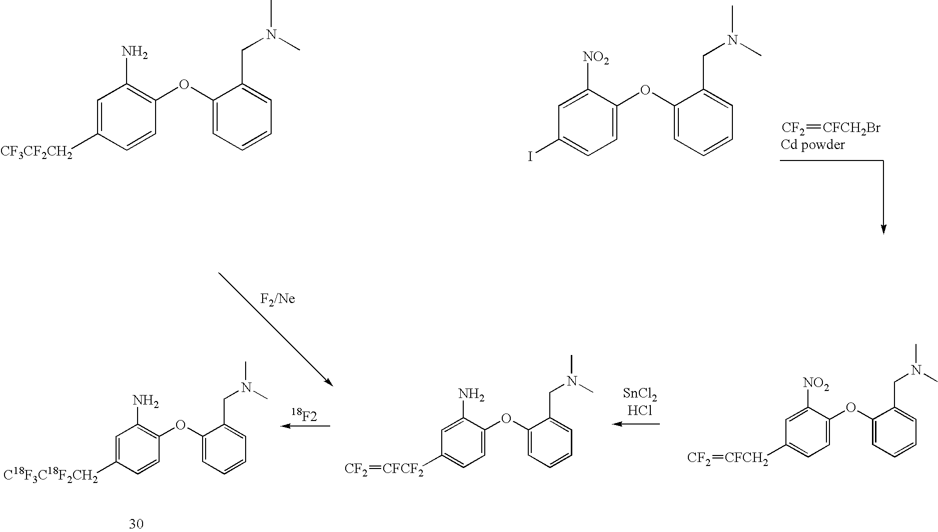

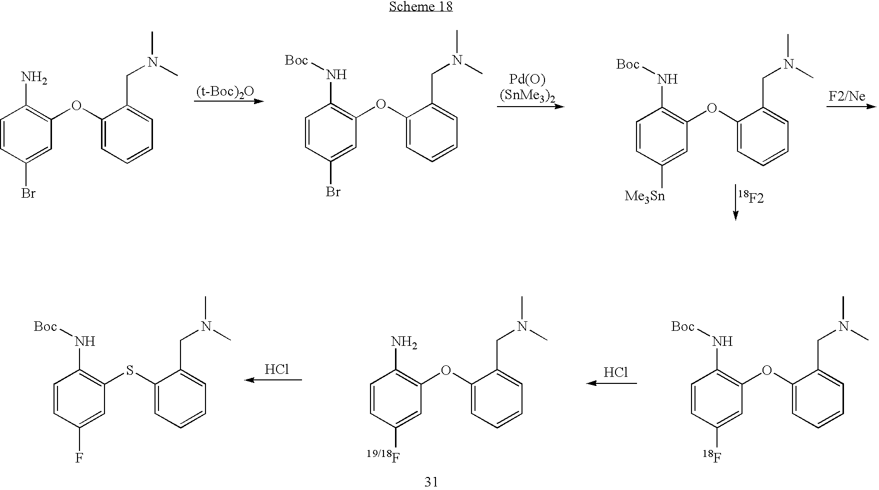

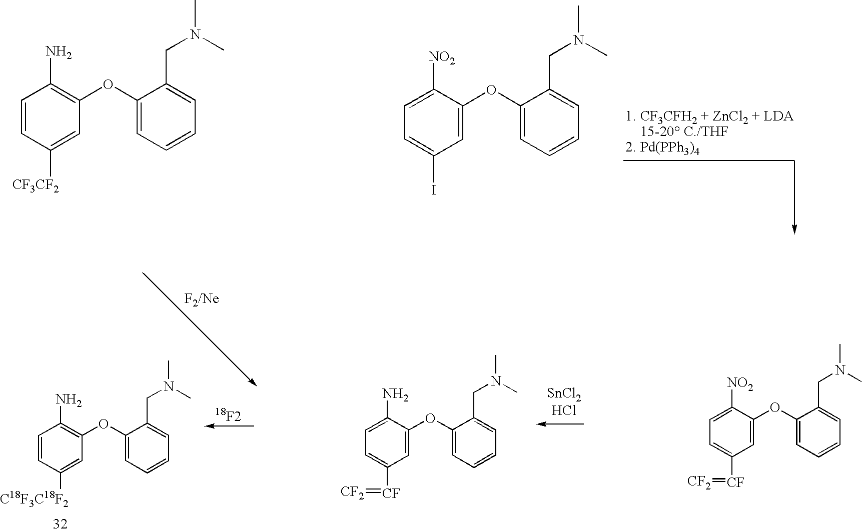

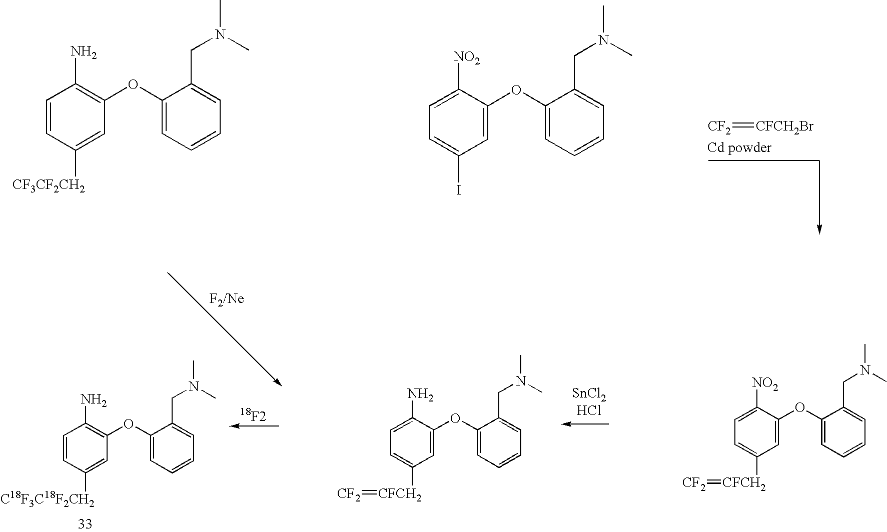

Schemes 21 through 26 depict synthetic routes for preparing compounds of Formula III.

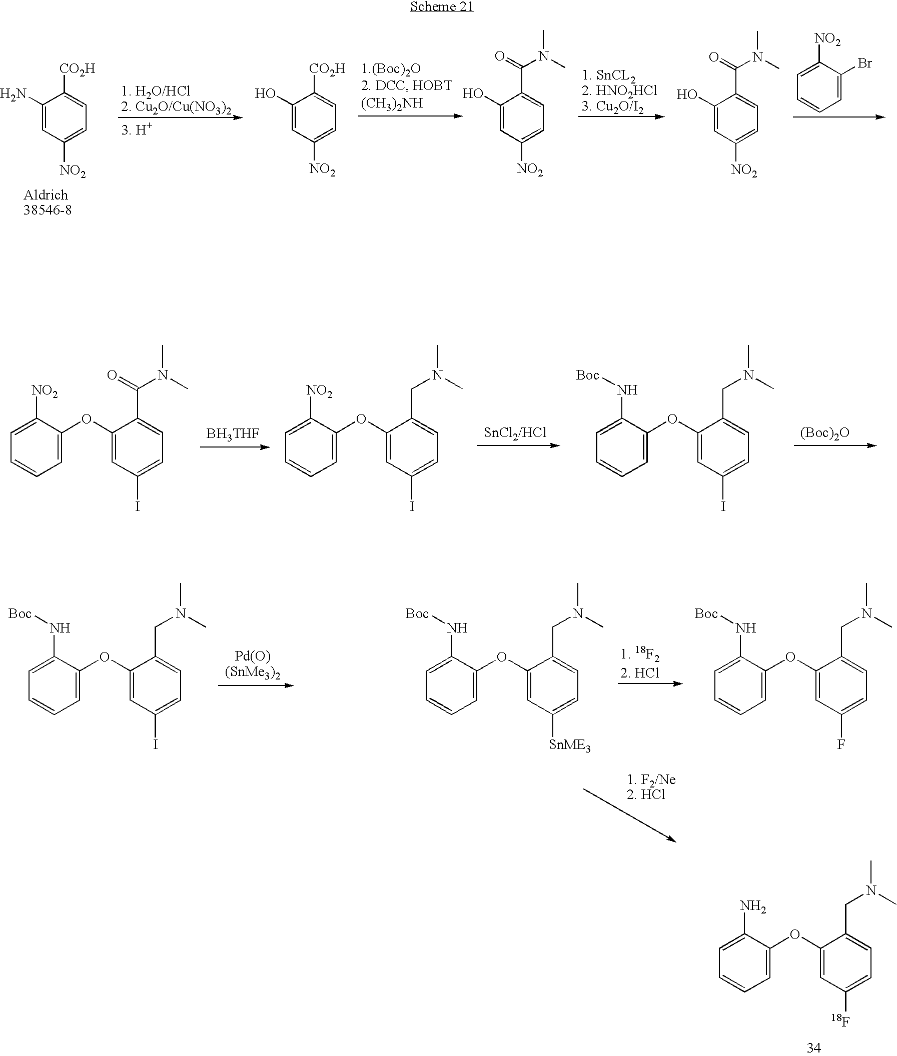

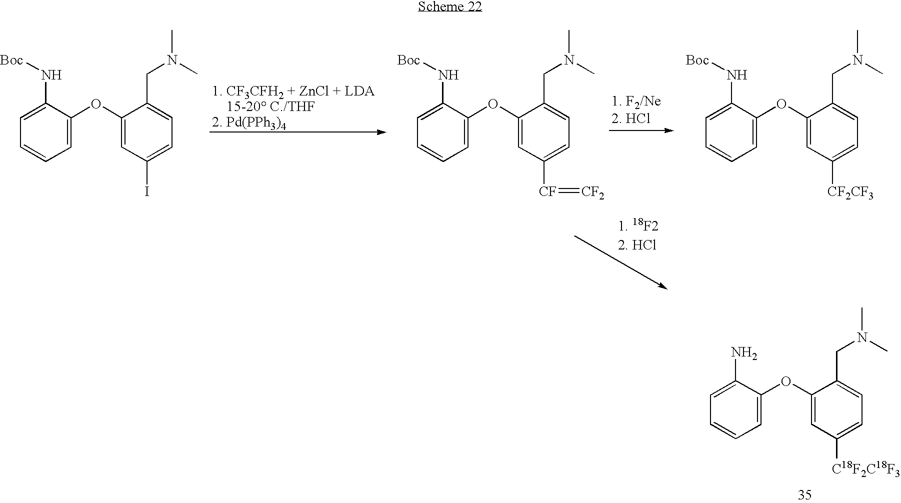

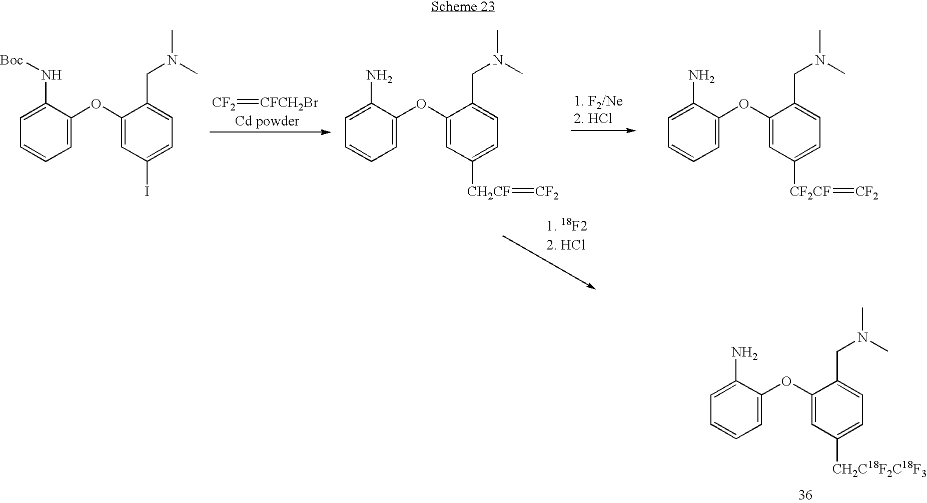

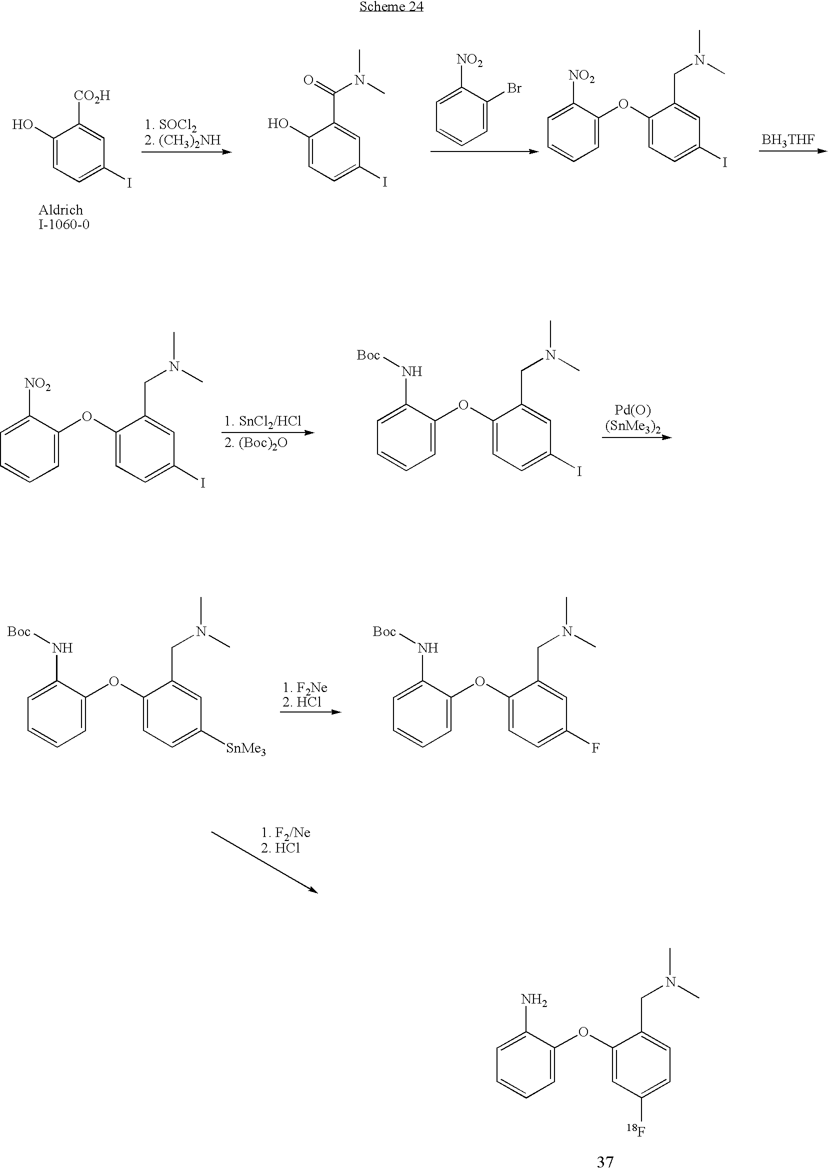

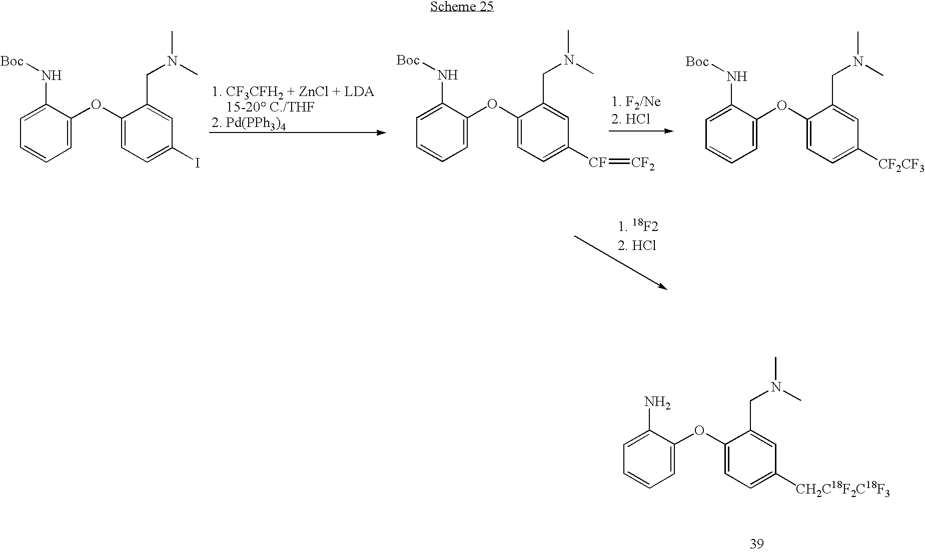

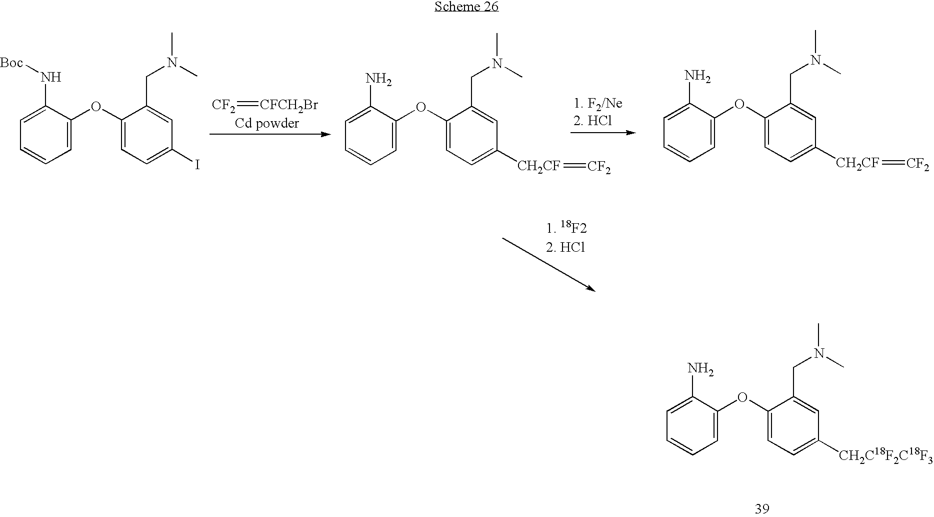

The radiohalogenated compounds of this invention lend themselves easily to formation from materials which could be provided to users in kits. Kits for forming the imaging agents can contain, for example, a vial containing a physiologically suitable solution of an intermediate of Formula I in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and transporters in the brain imaged by means of measuring the gamma ray or photon emissions therefrom.

When desired, the radioactive diagnostic agent may contain any additive such as pH controlling agents (e.g., acids, bases, buffers), stabilizers (e.g., ascorbic acid) or isotonizing agents (e.g., sodium chloride).

The term “pharmaceutically acceptable salt” as used herein refers to those carboxylate salts or acid addition salts of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci. 66:1-19 (1977).

In the first step of the present method of imaging, a labeled compound of Formula I is introduced into a tissue or a patient in a detectable quantity. The compound is typically part of a pharmaceutical composition and is administered to the tissue or the patient by methods well known to those skilled in the art.

For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray.

In a most preferred embodiment of the invention, the labeled compound is introduced into a patient in a detectable quantity and after sufficient time has passed for the compound to become associated with SERTs, the labeled compound is detected noninvasively inside the patient. Also contemplated is another embodiment of the invention wherein, a labeled compound of Formula I is introduced into a patient, sufficient time is allowed for the compound to become associated with SERTs, and then a sample of tissue from the patient is removed and the labeled compound in the tissue is detected apart from the patient. In a third embodiment of the invention, a tissue sample is removed from a patient and a labeled compound of Formula I is introduced into the tissue sample. After a sufficient amount of time for the compound to become bound to amyloid deposits, the compound is detected.

The administration of the labeled compound to a patient can be by a general or local administration route. For example, the labeled compound may be administered to the patient such that it is delivered throughout the body. Alternatively, the labeled compound can be administered to a specific organ or tissue of interest, such as the brain, where the compounds of the present invention associate selectively with SERTs.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with SERTs. The amount of time necessary can easily be determined by introducing a detectable amount of a labeled compound of Formula I into a patient and then detecting the labeled compound at various times after administration.

The term “associated” means a chemical interaction between the labeled compound and the SERT. Examples of associations include ligation, covalent bonds, ionic bonds, hydrophilic-hydrophilic interactions, hydrophobic-hydrophobic interactions, and complexes.

Those skilled in the art are familiar with the various ways to detect labeled compounds. For example, PET or SPECT can be used to detect radiolabeled compounds. The label that is introduced into the compound will depend on the detection method desired. For example, if PET is selected as a detection method, the compound must possess a positron-emitting atom, such as ¹¹C or ¹⁸F.

One of the key prerequisites for an in vivo imaging agent of the brain is the ability to cross the intact blood-brain barrier after a bolus iv injection. To test the permeability of compounds of Formula I, the compounds were injected into rats. Brain uptake was measured and the data are shown in Table 2.

The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

The following examples are illustrative, but not limiting, of the method and compositions of the present invention. Other suitable modifications and adaptations of the variety of conditions and parameters normally encountered and obvious to those skilled in the art are within the spirit and scope of the invention.

### EXAMPLES

Reagents and solvents were purchased from Aldrich and used without further purification except for tetrahydrofuran (THF) which was distilled immediately before use from sodium benzophenone. C₁₈ Sep-Pak cartridges were obtained from Waters Chromatography Division, Millipore Corporation. Radioactivity was determined using a calibrated ion chamber (Capintec CRC-745, Capintec, Inc.) and a sodium iodide well counter (Packard, Gamma Counter 5000 Series, Packard Instrument Company, IL).

High performance liquid chromatography (HPLC) analyses were carried out with a Sonntek liquid chromatograph equipped with both u.v. and radioactivity monitors. For the semi-preparative separations, a reversed phase C₁₈ column (10×250 mm, Phenomenex Luna (2)) was used with CH₃CN:0.1 M HCO₂NH₄ (30:70) containing 0.3 v % of acetic acid as the solvent with a flow rate of 5 ml/min. For the specific activity determinations, an analytical reversed phase C₁₈ column (4.6×250 mm, Phenomenex Luna (2)) was used with the same solvent as that used for the semi-preparative separations with a flow rate of 0.8 m/min.

The elemental analyses were performed by Atlantic Microlab, Inc. Norcross, Ga. Elemental compositions were within ±0.3% of the calculated values. Melting points were determined on a MEL-Temp II apparatus and are uncorrected. ¹H NMR spectra were recorded on a Bruker DPX 200 spectrometer. Chemical shifts (8) are expressed in parts per million relative to internal tetramethylsilane. Single-Crystal X-ray Crystallography of compounds 4 (C₁₅H₁₅SN₂O₂Br) and 5 (C₁₅H₁₈BSN₂O₂Br) were performed by X-Ray Diffraction Laboratory, Chemistry Department, University of Pennsylvania.

### Example 1

### [2-(4-Bromo-2-nitrophenoxy)benzyl]dimethylamine (8)

To a stirred solution of potassium hydroxide (741 mg, 13.2 mmol) in water (18.2 mL) was added 2-dimethylaminomethylphenol (6) (2.0 g, 13.2 mol). The resulting solution was stirred at room temperature for 30 munites and then concentrated to yield the phenoxide, which was used without further purification. DMSO (21 mL) was added to the phenoxide and then 2,5-dibromonitrobenzene (7) (5.1 g, 18.2 mmol) with stirring. The resulting mixture was heated to 120° C. for 8 h. The mixture was then cooled to room temperature and diluted with ethyl acetate and water. The aqueous phase was extracted three times with ethyl acetate and the combined organic extracts were washed with brine, dried with magnesium sulfate, filtered, and concentrated to yield a dark orange oil. The product was isolated by column chromatography using silica gel and a solvent system of 40% ethyl acetate:60% hexane. The R_{f} value of the product 8, in 40% ethyl acetate:60% hexane was 0.2. The reaction yielded compound 8 (3.7 g, 80%): IR (cm⁻¹, neat) 3375, 2941, 2817, 2768, 1602, 1581, 1530, 1475, 1349, 1246; ¹H NMR (500 MHz CDCl₃) δ 8.07 (d, J=2.3 Hz, 1H), 7.52-7.54 (m, 2H), 7.24-7.30 (m, 2H), 6.94 (d, J=8.2 Hz, 1H), 6.75 (d, J=8.2 Hz, 1H), 3.49 (s, 2H), 2.24 (s, 6H); HRMS calcd for C₁₅H₁₅N₂O₃Br (M⁺+1) 351.0266, found 351.0344.

### Example 2

### 5-Bromo-2-(2-dimethylaminomethyl)phenoxy)phenylamine (9)

To a suspension of 8 (760 mg, 2.17 mmol) in concentrated HCl (8 mL) and methanol (16 mL) was added SnCl₂ (1.6 g) at 0° C. The reaction mixture was stirred and warmed to room temperature. The reaction was stirred at room temperature overnight. The acidic solution was diluted with water (20 mL) and extracted one time with ethyl acetate. The organic layer was discarded. The aqueous layer was basified to pH=12 with 20% NaOH. The aqueous layer was then extracted three times with ethyl acetate. The combined organic extracts were then washed with brine, dried with magnesium sulfate, filtered, and concentrated to yield 9 (480 mg, 68%) as a brown solid. The R_{f} value of the product in 10% methanol:90% dichloromethane is 0.4: IR (cm⁻¹, neat) 3450, 3293, 3139, 2940, 2819, 2772, 2337, 1596, 1495, 1452, 1421, 1224, 1197; ¹H NMR (500 MHz CDCl₃) δ 7.26 (m, 1H), 7.17 (m, 1H), 7.02 (m, 1H), 6.78-6.82 (m, 41), 4.74 (broad s, 2H), 3.53 (s, 2H), 2.26 (s, 6H); HRMS calcd for C₁₅H₁₇N₂OBr (M⁺) 320.0524, found 320.0523.

### Example 3

### 2-(2-(Dimethylaminomethyl)phenoxy)-5-(tributyltin)phenylamine (10)

Compound 9 (101.8 mg, 0.32 mmol) was placed in a sealed tube with dioxane (6.7 mL) and triethylamine (1.7 mL). The solution was stirred at room temperature and bistribulyl tin (0.8 mL) was added followed by Pd(PPh₃)₄ (40 mg). The tube was sealed and the reaction mixture was heated to 125° C. with stirring. The reaction was monitored by TLC and was complete as indicated by the absence of starting material in 8 h. The crude reaction mixture was cooled to room temperature and solvent was removed in vacuo. The product was purified by silica gel preparative TLC twice using a solvent system of 10% methanol:90% dichloromethane. The R_{f} value of the product in that solvent system is 0.6. Compound 10 (68.7 mg, 43.4%) was isolated as a brown oil: IR (cm⁻¹, neat) 3308, 2954, 2923, 2851, 2356, 1582, 1486, 1453, 1227, 1197; ¹H NMR (500 MHz CDCl₃): δ 7.44 (d, J=7.5 Hz, 1H), 7.25 (m, 1H), 7.06 (m, 1H), 6.75-6.87 (m, 4H), 3.85 (s, 2H), 2.48 (s, 6H), 0.87-1.53 (m, 27H); HRMS calcd for C₂₇H₄₄N₂OSn (M⁺+1) 533.2553, found 533.2531.

### Example 4

### 2-(2-(Dimethylaminomethyl)phenoxy)-5-iodophenylamine (4)

To a solution of compound 10 (41.5 mg, 0.078 mmol) in CHCl₃ (5.9 mL) was added a solution of iodine in CHCl₃ (0.1 M) until the iodine color persisted. At this time the TLC showed no starting material. The reaction mixture was treated with KF in methanol (1.0 M, 0.2 mL) and stirred with sodium bisulfite (5%, 0.5 mL) for 5 min. The reaction mixture was diluted with water and chloroform. Two layers were separated and the aqueous layer was discarded. The organic layer was washed with brine, dried with magnesium sulfate, filtered, and concentrated. The crude reaction was purified by silica gel preparative TLC to yield 4 (21.3 mg, 74%) as a brown solid. Compound 4 has an R_{f} value of 0.5 in 10% methanol:90% dichloromethane: IR (cm⁻¹, neat) 3317, 2952, 2921, 2851, 2358, 1592, 1492, 1461, 1413, 1225, 1196; ¹H NMR (500 MHz CDCl₃) δ 7.31 (d, J=7.5 Hz, 1H), 7.20 (m, 1H), 7.02-7.08 (m, 2H), 6.96 (d, J=8.0 Hz, 111), 6.81 (d, J=8.0 Hz, 1H), 6.61 (d, J=8.0 Hz, 1H), 4.65 (broad s, 2H), 3.60 (s, 2H), 2.32 (s, 6H); HRMS calcd for C₁₅H₁₇N₂OI (++1) 369.0385, found 369.0462.

### Example 5

### ([¹²⁵I][¹²³I]2-(2-(Dimethylaminomethyl)phenoxy)-5-iodophenylamine ([¹²⁵I]/[¹²³I]4)

The tin compound 10 (50 μg in 50 μL of ethanol), [I-125]/[I-123] sodium iodide, and 1N HCl (100 μL) were placed in a sealed vial. To this mixture, 100 μL of H₂O₂ (3% solution in water) was added via a syringe at room temperature. The iodination reaction was terminated after 10 min by an addition of saturated NaHSO₃ and the resulting solution was neutralized by adding a saturated NaHCO₃ solution. The mixture was purified by a C4 mini-column method (yield >90%). The purity was determined by HPLC (PRP-1 column, acetonitrile/5 mM 3,3-dimethylglutaric acid, pH 7.0:90/10, flow rate=1 mL/min; retention time=9.3 min). Radiochemical purity was 99%.

### Example 6

### (4-Bromo-2-nitrophenyl)(2-dimethylaminomethylphenyl)methanol (13a)

A solution of 2-bromo-N,N-dimethylbenzylamine (11) (268 mg, 1.25 mmol) in THF (5 mL) was added to a solution of BuLi (1.7 mL, 1.6 M in THF, 2.7 mmol) in THF (5 mL) dropwise at RT. The mixture was stirred at RT for 10 min The resulting lithium species was added to a solution of 4-bromo-2-nitrobenzaldehyde (12a) (311 mg, 1.35 mmol) in THF (5 mL) dropwise at −78° C. The mixture was stirred at RT overnight. Ice water was added and the mixture was extracted with CH₂Cl₂. The organic phase was dried under Na₂SO₄, filtered and the filtrate was concentrated to give crude product which was purified by PTLC (hexane/ethyl acetate (1/1)) to give 60 mg of product (12%): ¹H NMR (200 MHz, CDCl₃) δ 2.28 (s, 6H), 3.11 (d, J=12.5 Hz, 1H), 4.14 (d, J=12.5 Hz, 1H), 6.56 (s, 1H), 6.58 (d, J=8.8 Hz, 1H), 7.07-7.24 (m, 3H), 7.86 (d,d, J=8.5, 2.2 Hz, 1H), 8.05 (d, J=8.5 Hz, 1H), 8.16 (d, J=2.0 Hz, 1H).

### Example 7

### (2-Dimethylaminomethylphenyl)(4-iodo-2-nitrophenyl)methanol (13b)

The same procedure described above for preparation of 13a was employed to prepare 13b. Product 13b was obtained (68 mg, 16%) from 4-iodo-2-nitrobenzaldehyde (12b) (277 mg, 1 mmol): ¹H NMR (200 MHz, CDCl₃) δ 2.32 (s, 6H), 3.15 (d, J=12.5 Hz, 1H), 4.31 (d, J=12.5 Hz, 1H), 6.03 (s, 1H), 6.41 (d, J=7.2 Hz, 1H), 7.09-7.29 (m, 3H), 8.02 (d, J=8.5, Hz, 1H), 8.39 (d,d, J=6.8, 2.3 Hz, 1H), 8.66 (d, J=2.3 Hz, 1H).

### Example 8

### 5-Bromo-2-(2-(dimethylaminomethyl)benzyl)phenylamine (14)

To a solution of nitro compound (13a) (67 mg, 0.18 mmol) in THF (5 mL) was added a solution of BH₃ (2 mL, IM in THF) dropwise at RT. The resulting mixture was stirred under reflux overnight. Water was slowly to neutralize the excess of BH₃. The solvent was removed on the Rotavapor. HCl solution (20 mL, 2% solution) was added and the mixture was refluxed for 30 min NH₄OH (conc.) was added after cooling to adjust the pH of the solution to basic. The mixture was extracted with mixed solvent (CH₂Cl₂/MeOH (95/5)). The organic phase was dried under Na₂SO₄, filtered and the filtrate was concentrated to give crude product which was purified by PTLC (hexane/ethyl acetate (1/1)) to give 34 mg of product (58%): ¹H NMR (200 MHz, CDCl₃) δ 2.25(s, 6H), 3.47 (s, 2H), 3.90 (s, 2H), 6.71 (d, J=2.0 Hz, 1H), 6.80 (d,d, J=8.0, 2.0 Hz, 1H), 6.96 (d, J=7.9 Hz, 1H), 7.03-7.08 (m, 1H), 7.12-7.20 (m, 3H).

### Example 9

### 2-(2-(dimethylaminomethyl)benzyl)-5-iodophenylamine (5)

The same procedure described above for preparation of 14 was employed to prepare 5. Product 5 (13 mg, 21%) was obtained from 13b (68 mg, 0.16 mmol): ¹H NMR (200 MHz, CDCl₃) δ 2.23 (s, 6H), 3.34 (s, 2H), 4.00 (s, 2H), 6.54 (d,d, J=8.2, 2.3 Hz, 1H), 6.62 (d, J=8.2 Hz, 1H), 6.97 (m, 1H), 7.13-7.25 (m, 3H), 7.32-7.40 (m, 1H).

### Example 10

### 2-(2-(Dimethylaminomethyl)benzyl)-5-(tributyltin)phenylamine (15)

The mixture of bromide 14 (14 mg, 0.04 mmol), bistributyltin (0.1 mL), Pd(Ph₃)₄ (10 mg) and triethylamine (0.1 mL) in dioxane (2 mL) was stirred at 90° C. overnight. Solvent was removed and the residue was purified by PTLC (hexane/ethyl acetate (1/1)) to give 6 mg of product, 15 (26%): ¹H NMR (200 MHz, CDCl₃): δ 0.92 (t, J=7.8 Hz, 9H), 1.26-2.03 (m, 18H), 2.26 (s, 6H), 3.49 (s, 2H), 3.96 (s, 2H), 6.69 (s, 1H), 6.78 (d, J=7.1 Hz, 1H), 7.02 (d, J=7.0 Hz, 1H), 7.05-7.20 (m, 4H).

### Example 11

### 2-(2-(Dimethylaminomethyl)benzyl)-5-[¹²⁵I]iodophenylamine ([¹²⁵I]]5)

The tin compound 17 (50 μg in 50 μL of ethanol), [I-125]/[I-123] sodium iodide, and 1N HCl (100 μL) were placed in a sealed vial. To this mixture, 100 μL of H₂O₂ (3% solution in water) was added via a syringe at room temperature. The iodination reaction was terminated after 10 min by an addition of saturated NaHSO₃. The resulting solution was neutralized by adding a saturated NaHCO₃ solution. The mixture was loaded on a C4 mini-column. The cartridge was first washed with water. The desired product was washed off the cartridge by ethanol. Radiochemical purity after the purification was 99%.

### Example 12

Preparation of membrane homogenates: LLC-PK₁ cells from pig kidney permanently expressing DAT, NET and SERT, respectively (LLC-DAT, LLC-NET and LLC-SERT) were kindly provided by Dr. G. Rudnick at Yale University. These cells were grown to confluence as a monolayer on 15 cm petri dish as reported previously. (Gu, H., Wall, S. C. and Rudnick G., Journal of Biological Chemistry, 269: 7124-7130 (1194)). Cells were then washed once with phosphate buffer saline (w/ Ca²⁺ and Mg²⁺) (Bio-Whittaker) and harvested with 10 mL PBS by scraping. The cell suspensions were homogenized on ice for 20 strokes and centrifuged at 17,000 rpm for 20 min at 4° C. The pellets were re-suspended in PBS, frozen quickly in liquid N₂ and stored in a −70° C. freezer.

### Example 13

Binding assays: Binding assays were performed in a final volume of 0.2 mL. Aliquots of membrane suspensions (100 μL, corresponding to 30-40 g protein) were mixed with 50 mM Tris-HCl, pH 7.4, 120 mM NaCl and 0.1% BSA (all from Sigma, St. Louis, Mo.), 0.4 nM [¹²⁵I]IPT or [¹²⁵I]IDAM, and 8-10 concentrations (10⁻¹⁰ to 10⁻⁵ M) of competing drugs. Nonspecific binding was defined with 10 μM CFT for [¹²⁵I]IPT assays and 1 μM IDAM for [¹²⁵I]IDAM assays. Incubation was carried out for one h at room temperature and the bound ligand was collected on glass fiber filters (Schleicher & Schuell No. 25, Keene, N.H.) presoaked with 1% polyethylenimine (Sigma, St. Louis, Mo.) and counted in a gamma counter (Packard 5000). Results of competition experiments were subjected to nonlinear regression analysis using EBDA (Elsevier-BIOSOFT, Cambridge, UK).

Using an in vitro binding assay with membrane preparations containing the specific SERT expressed in LLC-PK₁ cells, 4 displayed good affinity to SERT sites showing a Kᵢ of 0.37±0.11 nM, which is more than 500 fold selective for SERT over NET and DAT (Kᵢ=286±36 and 309±65 nM, for NET and DAT, respectively). Using the same cell line expressing SERT, 5 exhibited a moderate binding affinity for SERT (Kᵢ=48.6±4.1 nM) and showing less selectivity between SERT and NET (Kᵢ=17±4 for NET). Unexpectedly, a slight modification of the bridgehead atom between the two phenyl groups results in a dramatic shift in selective binding between SERT and NET.

Table 1. Selectivity of compounds for monoamine transporters: SERT (serotonin transporter), DAT (dopamine transporter) or NET (norepinephrine transporter) (K_{I}, nM).

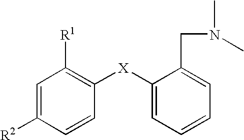

| | | X | R₁ | R₂ | SERT | DAT | NET |
|---|---|---|---|---|---|---|---|
| 1. | IDAM | S | CH₂OH | I | 0.097 ± 0.013 | >1,000 | 234 ± 26 |
| 2. | ODAM | O | CH₂OH | I | 0.12 ± 0.02 | >1,000 | 20 ± 2 |
| 3. | ADAM | S | NH₂ | I | 0.013 ± 0.003 | 840 ± 100 | 699 ± 80 |
| 4. | | O | NH₂ | I | 0.37 ± 0.11 | 309 ± 65 | 286 ± 36 |
| 5. | | CH₂ | NH₂ | I | 48.6 ± 4.10 | >1,000 | 17 ± 4 |
| | DASB | S | NH₂ | CN | 1.1 ± 0.04 | >1,000 | >1,000 |

| | | | |
|---|---|---|---|
| (+)McN5652 | 0.009 ± 0.002 | 112 ± 23 | 11.3 ± 3.3 |
| Nisoxetine | 203 ± 4 | >1,000 | 1.6 ± 0.14 |

### Example 14

Partition coefficients: Partition coefficients were measured by mixing [¹²⁵I] compound with 3 g each of 1-octanol and buffer (pH 7.4, 0.1 M phosphate) in a test tube. The test tube was vortexed for 3 min at room temperature, then centrifuged for 5 min. Two weighed samples (2 g each) from the 1-octanol and buffer layers were counted in a well counter. The partition coefficient was determined by calculating the ratio of cpm/g of 1-octanol to that of buffer. Samples from the 1-octanol layer were re-partitioned until consistent partition coefficient values were obtained. The measurement was repeated 3 times.

Partition coefficient of 4 was determined to be 418 between 1-octanol/buffer. The value is comparable to that of [¹²⁵I]ADAM (3) measured under the same conditions (P.C.=335) indicating both ligands have comparable lipophilicity. The partition coefficient determined for [¹²⁵I]5 was 1,730 between 1-octanol/buffer.

### Example 15

Biodistribution in rats: Three rats per group were used for each biodistribution study. While under ether anesthesia, 0.2 mL of a saline solution containing 10 μCi of radioactive tracer was injected into the femoral vein. The rats were sacrificed at the time indicated by cardiac excision while under ether anesthesia. Organs of interest were removed and weighed, and the radioactivity was counted. The percent dose per organ was calculated by a comparison of the tissue counts to counts of 1% of the initial dose (100 times diluted aliquots of the injected material) measured at the same time.

Regional brain distribution in rats was measured after an i.v. injection of the radioactive tracer. Samples from different brain regions: cortex (frontal plus occipital), striatum, hippocampus, cerebellum and hypothalamus were dissected, weighed and counted. The percentage dose/g of each sample was calculated by comparing sample counts with the counts of the diluted initial dose described above. The specific binding ratio in each region was obtained by dividing the % dose/gram values between region by that in the cerebellum ([region-cerebellum/cerebellum]). The cerebellum region containing little or no serotonin transporters was used as the background region.

In vivo competitive binding of various compounds in the regional uptake of [¹²⁵I]4 was investigated by pre-treating animals with various competing drugs (2 mg/kg, i.v. at 5 min prior to injection of [¹²⁵I]4). The competing drugs included ketanserin, raclopride, methylphenidate, nisoxetine, (+)McN5652, escitalopram and IDAM. Similar regional brain distributions were determined at 120 min after [¹²⁵I]4 injection as described above. The reduction of regional specific binding in the drug-pretreated rats was compared to the control animals with saline pretreatment.

Biodistribution of [¹²⁵I]4 in the rats showed a high initial uptake in the brain (1.77% dose at 2 min after i.v. injection) (Table 2). The total brain uptake did not decrease in the initial 60 min and then dropped significantly after 120 min. The hypothalamus region, a SERT rich area of the brain, showed high uptake and retention ([hypothalamus-cerebellum]/cerebellum ratio was 1.92, 3.49, 4.49 and 2.64 at 60, 120, 240 and 360 min after injection, respectively) (Table 2). Comparable values ([hypothalamus-cerebellum]/cerebellum ratios) were observed for [¹²⁵I]ADAM (FIG. 4). Biodistribution of [¹²⁵I]4 in other organs or tissues was similar to that reported previously for [¹²⁵I]ADAM (3). (Oya, S., et al., Nuclear Medicine and Biology, 27: 249-254 (2000); Choi, S. R., et al., Synapse, 38: 403-412 (2000)). Interestingly, the less potent and selective SERT ligand, [¹²⁵I]5 exhibited a similar initial brain uptake (1.77% dose at 2 min postinjection) but with a fast kinetic washout as compared to [¹²⁵I]4 The highest specific binding in hypothalamus ([hypothalamus-cerebellum]/cerebellum) at 120 min post-injection was found to be 2.43. Likely, this specific signal could be due to the concomitant contamination of NET with SERT for [¹²⁵I]5.

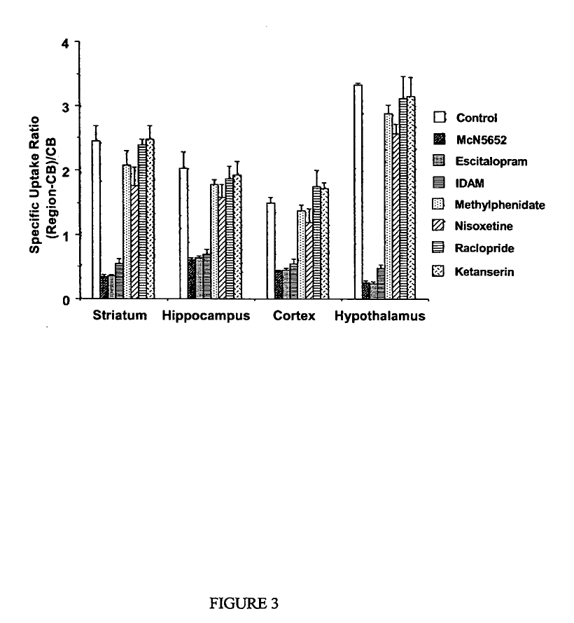

A surprising and unexpected observation for this series of compounds as SERT specific imaging agents was the high brain uptake of 4, exhibited at 2 and 4 h after i.v. injection (1.08 and 0.33 vs 0.42 and 0.11% dose/brain for 4 vs ADAM (3)). This represents a two to three-fold increase in the brain uptake. It is important to note that between 2-4 h after an i.v. injection is the time range important for in vivo imaging studies; therefore, the increase in uptake will significantly benefit the imaging study. Significantly, the target area in the brain, i.e. hypothalamus, where SERT density was the highest; 4 displayed more than 200% higher uptake in the hypothalamus between 2 and 4 h than that of ADAM (3) (0.92 and 0.32 vs 0.44 and 0.13% dose/g for 4 and ADAM (3), respectively). Compound 4 exhibits the highest hypothalamus uptake among all of the radioiodinated compounds in this series of SERT imaging agents (FIG. 3A). However, the washout rate of [¹²⁵I]4 from cerebellum, a non-SERT containing region, was relatively slow. Thus, the specific uptake ratio [(hypothalamus-cerebellum)/cerebellum] between 2-4 h showed a similar ratio for 4 and ADAM (3) (FIG. 3B). The higher SERT uptake can provide improved count rates for imaging studies; therefore, compound 4 exhibits unexpected and promising properties.

TABLE 2
Biodistribution in rats after an iv injection of [¹²⁵I]4 (% dose/organ, avg of 3 rats ± SD)

| Organ | 2 min | 30 min | 60 min | 120 min | 240 min | 6 hrs | 11 hrs | 24 hrs |
|---|---|---|---|---|---|---|---|---|
| Blood | 5.75 ± 0.54 | 4.56 ± 0.20 | 3.89 ± 0.28 | 3.15 ± 0.44 | 3.12 ± 0.32 | 3.29 ± 0.27 | 2.52 ± 0.47 | 0.70 ± 0.06 |
| Heart | 1.51 ± 0.13 | 0.32 ± 0.04 | 0.20 ± 0.01 | 0.13 ± 0.01 | 0.08 ± 0.02 | 0.08 ± 0.00 | 0.06 ± 0.00 | 0.02 ± 0.00 |
| Muscle | 25.9 ± 4.82 | 18.1 ± 0.55 | 12.9 ± 0.84 | 9.18 ± 0.65 | 6.27 ± 0.06 | 5.01 ± 0.42 | 4.01 ± 0.85 | 1.13 ± 0.13 |
| Lung | 17.2 ± 2.32 | 3.56 ± 0.96 | 1.79 ± 0.06 | 0.88 ± 0.04 | 0.37 ± 0.03 | 0.25 ± 0.01 | 0.16 ± 0.00 | 0.04 ± 0.00 |
| Kidney | 4.27 ± 0.90 | 2.67 ± 0.48 | 1.67 ± 0.21 | 1.24 ± 0.15 | 0.61 ± 0.09 | 0.55 ± 0.07 | 0.40 ± 0.10 | 0.17 ± 0.02 |
| Spleen | 0.44 ± 0.08 | 0.84 ± 0.05 | 0.47 ± 0.02 | 0.27 ± 0.01 | 0.13 ± 0.02 | 0.11 ± 0.01 | 0.09 ± 0.01 | 0.05 ± 0.01 |
| Liver | 5.71 ± 0.88 | 7.10 ± 0.66 | 5.79 ± 0.70 | 4.90 ± 0.38 | 3.52 ± 0.20 | 3.59 ± 0.23 | 2.64 ± 0.19 | 1.83 ± 0.07 |
| Skin | 4.45 ± 0.52 | 11.3 ± 0.78 | 13.9 ± 1.00 | 12.5 ± 2.14 | 12.6 ± 0.48 | 15.5 ± 1.50 | 10.9 ± 4.62 | 4.02 ± 0.26 |
| Brain | 1.77 ± 0.07 | 2.12 ± 0.15 | 1.74 ± 0.08 | 1.08 ± 0.06 | 0.33 ± 0.02 | 0.11 ± 0.01 | 0.02 ± 0.00 | 0.01 ± 0.00 |
| Thyroid | 0.10 ± 0.02 | 0.15 ± 0.04 | 0.41 ± 0.11 | 0.96 ± 0.06 | 2.34 ± 1.16 | 2.68 ± 0.66 | 5.15 ± 1.49 | 4.61 ± 1.21 |
| Region | 2 min | 30 min | 60 min | 120 min | 240 min | 6 hrs | 11 hrs | 24 hrs |

Regional brain distribution (% dose/g)::
CB; Striatum: ST; Hippocampus: HP; Cortex: CX; Hypothalamus: HY

| | | | | | | | | |
|---|---|---|---|---|---|---|---|---|
| CB | 0.79 ± 0.21 | 0.64 ± 0.09 | 0.41 ± 0.03 | 0.21 ± 0.01 | 0.06 ± 0.00 | 0.027 ± 0.002 | 0.011 ± 0.0013 | 0.0049 ± 0.0009 |
| ST | 0.77 ± 0.04 | 1.16 ± 0.10 | 1.02 ± 0.11 | 0.72 ± 0.01 | 0.24 ± 0.02 | 0.068 ± 0.005 | 0.012 ± 0.0000 | 0.0039 ± 0.0004 |
| HP | 0.86 ± 0.07 | 1.06 ± 0.12 | 0.94 ± 0.08 | 0.65 ± 0.04 | 0.20 ± 0.02 | 0.059 ± 0.005 | 0.012 ± 0.0021 | 0.0040 ± 0.0005 |
| CX | 1.16 ± 0.14 | 1.25 ± 0.13 | 0.95 ± 0.07 | 0.51 ± 0.04 | 0.12 ± 0.01 | 0.042 ± 0.002 | 0.010 ± 0.0010 | 0.0040 ± 0.0007 |
| HY | 0.96 ± 0.15 | 1.30 ± 0.06 | 1.18 ± 0.08 | 0.92 ± 0.06 | 0.32 ± 0.03 | 0.097 ± 0.015 | 0.016 ± 0.0025 | 0.0060 ± 0.0010 |

Ratio of (Region-Cerebellum)/Cerebellum Cerebellum

| | | | | | | | | |
|---|---|---|---|---|---|---|---|---|
| ST | 0.01 ± 0.27 | 0.83 ± 0.23 | 1.51 ± 0.12 | 2.53 ± 0.17 | 3.11 ± 0.35 | 1.56 ± 0.41 | 0.14 ± 0.13 | — |
| HP | 0.13 ± 0.22 | 0.68 ± 0.22 | 1.30 ± 0.05 | 2.19 ± 0.07 | 2.47 ± 0.28 | 1.21 ± 0.11 | 0.09 ± 0.08 | — |
| CX | 0.56 ± 0.55 | 0.96 ± 0.14 | 1.35 ± 0.07 | 1.48 ± 0.13 | 1.12 ± 0.17 | 0.58 ± 0.09 | 0.00 ± 0.16 | — |
| HY | 0.24 ± 0.17 | 1.06 ± 0.22 | 1.92 ± 0.20 | 3.49 ± 0.09 | 4.49 ± 0.12 | 2.64 ± 0.54 | 0.44 ± 0.19 | 0.23 ± 0.15 |

### Example 16

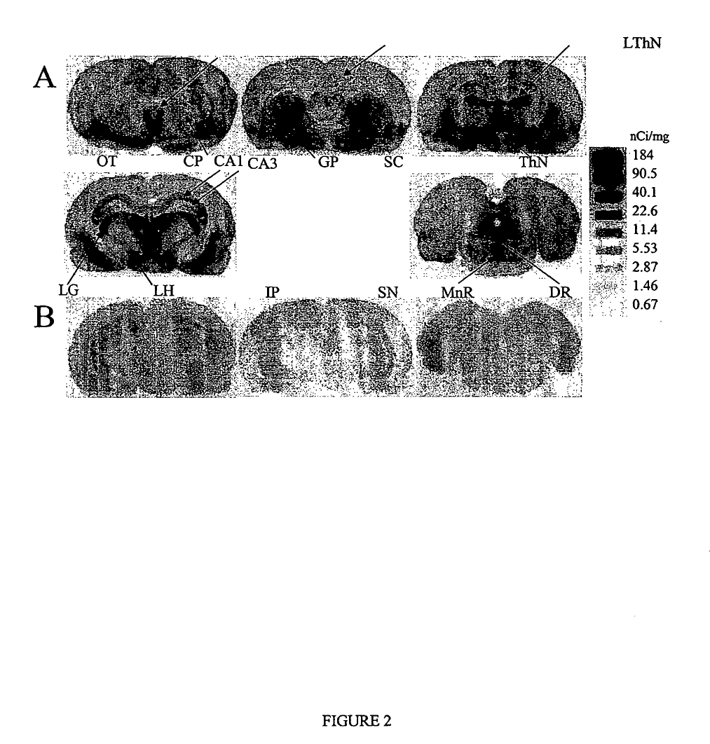

Autoradiography: Ex vivo autoradiography of [¹²⁵I]4 at 2 hr after an i.v. injection into rats showed a distinctive distribution pattern in the brain, which reflected regional distribution of SERT binding sites, Intense labeling of olfactory tubercle (OT), lateral hypothalamic (IL) and thalamic nuclei (ThN), globus pallidus (GP), superior colliculus (SC), substantia nigra (SN), interpeduncular nucleus (P), dorsal (DR) and median raphes (MnR) (FIG. 2A), areas known to have high densities of SERT sites. (Cortes, R., et al., Neuroscience, 27: 473-496 (1988); Biegon, A., et al., Brain Research, 619: 236-246 (1993)) Lower but detectable labeling was also found in frontal cortex (Cx), caudate putamen (CP), ventral pallidum and hippocampus, areas containing a significantly lower amount of SERT sites. The regional distribution is consistent with those reported for other SERT ligands, [¹²⁵I]ADAM (3) (Choi, et al.) paroxetine, (Cortes, et al.) (+)McN5652 (Suchiro, M., et al., Nuclear Medicine and Boilogy, 21: 1083-1091 (1994); (Scheffel, U., et al., NIDA Research Monograph, 138: 111-130 (1994) and [¹²⁵I]5-Iodo-6-nitroquipazine. (Biegon, et al.) After a rat was pretreated with a dose of (+)McN5652 (2 mg/Kg body weight) at 5 min prior to the i.v. injection of [¹²⁵I]4, the specific uptake in the regions of the brain was totally eliminated suggesting that these two agents are competing for the same SERT binding sites (FIG. 2B). The blocking study by ex vivo autoradiography lends support to the contention that this tracer binds to the SERT binding sites and the binding can be blocked by a chemical dose of a competing drug, (+)McN5652.

### Example 17

Blocking studies: Blocking studies with selective SERT ligands, i.e. citalopram and (+)McN5652, completely eliminated the specific binding in the hypothalamus region indicating the in vivo [¹²⁵I]4 binding to SERT (FIG. 3). Non-SERT agents, i.e. methylphenidate (DAT), nisoxetine (NET), raclopride (D2/D3) and ketanserin (5-HT_{2A}) did not show any significant effect on the specific uptake. The data suggest that this tracer is highly selective in binding to the SERT binding sites in vivo. The SPECT images obtained approximately at the level of the midbrain at 180-240 min post injection were very similar to those obtained with [¹²³I]ADAM (3).

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A compound of Formula I 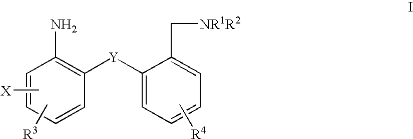
or a pharmaceutically acceptable salt thereof; wherein,
Y is O or CH₂,
one of R¹ and R² is hydrogen or C₁₋₄ alkyl, the other of R¹ or R² is C₁₋₄ alkyl,
R³ is selected from the group consisting of hydrogen and C₁₋₄ alkyl,
R⁴ is selected from the group consisting of hydrogen, C₁₋₄ alkyl and halo(C₁₋₄)alkyl, and
X is ¹³¹I, ¹²³I, ¹²⁵I, ¹⁸F, ⁷⁶Br, ⁷⁷Br, ¹⁸Fluoro(C₁₋₄)alkyl; or CF₃CF₂(CH₂)ₙ—, wherein at least one F is ¹⁸F, and n is zero or one.

2. The compound of claim 1, wherein
R¹ is hydrogen.

3. The compound of claim 1, wherein
R¹ is methyl.

4. The compound of claim 3, wherein
R² is methyl.

5. The compound of claim 1, wherein
R³ is hydrogen.

6. The compound of claim 5, wherein
R⁴ is hydrogen.

7. The compound of claim 1, wherein
Y is O.

8. The compound of claim 7, wherein
R¹ is hydrogen.

9. The compound of claim 7, wherein
R² is methyl.

10. The compound of claim 7, wherein
R³ is hydrogen.

11. The compound of claim 10, wherein
R⁴ is hydrogen.

12. The compound of claim 11, wherein
R¹ and R² are each C₁₋₄ alkyl.

13. The compound of claim 12, wherein
R¹ and R² are each methyl.

14. The compound of claim 13, wherein
X is selected from the group consisting of ¹³¹I, ¹²³I, ¹²⁵I, ¹⁸F and CF₃CF₂(CH₂)ₙ—, wherein at least one F is ¹⁸F, and n is zero or one.

15. The compound of claim 14 having the formula: 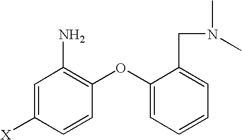
wherein X is ¹³¹I, ¹²³I or ¹²⁵I.

16. The compound of claim 14 having the formula: 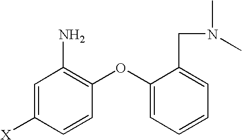
wherein X is ¹⁸F.

17. The compound of claim 14 having the formula: 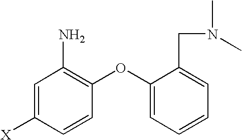
wherein X is CF₃CF₂(CH₂)ₙ—, wherein at least one F is ¹⁸F, and n is zero or one.

18. The compound of claim 14 having the formula:

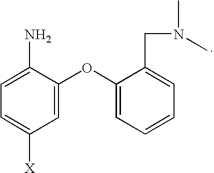

19. The compound of claim 14 having the formula:

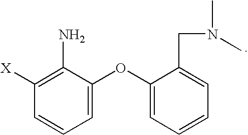

20. The compound of claim 18 or 19 wherein, X is CF₃CF₂(CH₂)ₙ—, wherein at least one F is ¹⁸F, and n is zero or one.

21. A compound of Formula II 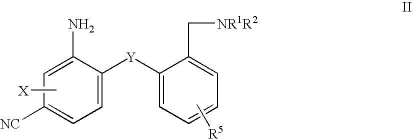
or a pharmaceutically acceptable salt thereof; wherein,
Y is O or CH₂,
one of R¹ and R² is hydrogen or C₁₋₄ alkyl, the other of R¹ or R² is C₁₋₄ alkyl,
R⁵ is selected from the group consisting of hydrogen, C₁₋₄ alkyl and halo(C₁₋₄)alkyl, and
X is ¹¹³I, ¹²³I, ¹²⁵I, ¹⁸F, ⁷⁶Br, ⁷⁷Br, ¹⁸Fluoro(C₁₋₄)alkyl; or CF₃CF₂(CH₂)ₙ—, wherein at least one F is ¹⁸F, and n is zero or one.

22. The compound of claim 21, wherein
Y is O, and
X is ¹⁸F or CF₃CF₂(CH₂)ₙ—, wherein at least one F is ¹⁸F, and n is zero or one.

23. The compound of claim 22, wherein
X is CF₃CF₂(CH₂)ₙ—, wherein at least one F is ¹⁸F, and n is zero.

24. The compound of claim 23, wherein
X is CF₃CF₂(CH₂)ₙ—, wherein at least one F is ¹⁸F, and n is one.

25. The compound of claim 21 having the formula: 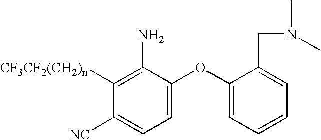
wherein at least one F is ¹⁸F, and n is zero or one.

26. The compound of claim 21 having the formula: 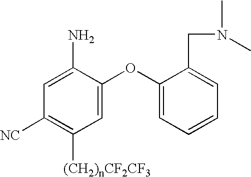
wherein at least one F is ¹⁸F, and n is zero or one.

27. The compound of claim 21 having the formula:

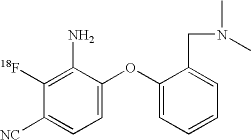

28. The compound of claim 21 having the formula:

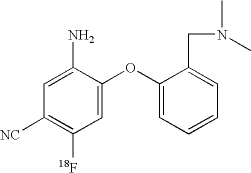

29. A compound of Formula III 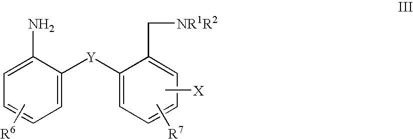
or a pharmaceutically acceptable salt thereof; wherein,
Y is O or CH₂,
one of R¹ and R² is hydrogen or C₁₋₄ alkyl, the other of R¹ or R² is C₁₋₄ alkyl,
R⁶ is selected from the group consisting of hydrogen, C₁₋₄ alkyl and halo(C₁₋₄)alkyl,
R⁷ is selected from the group consisting of hydrogen, C₁₋₄ alkyl, cyano, ¹⁸F and CF₃CF₂(CH₂)ₙ—, wherein at least one F is ¹⁸F, and n is zero or one, and
X is hydrogen, cyano, ¹³¹I, ¹²³I, ¹²⁵I, ¹⁸F, ⁷⁶Br, ⁷⁷Br, ¹Fluoro(C₁₋₄)alkyl; or CF₃CF₂(CH₂)ₙ—, wherein at least one F is ¹⁸F, and n is zero or one.

30. The compound of claim 29 having the formula:

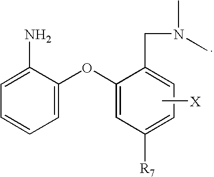

31. The compound of claim 30 wherein,
R⁷ is selected from the group consisting of ¹⁸F, cyano and CF₃CF₂(CH₂)ₙ—, wherein at least one F is ¹⁸F, and n is zero or one, and
X is selected from the group consisting of ¹⁸F, cyano and CF₃CF₂(CH₂)ₙ—, wherein at least one F is ¹⁸F, and n is zero or one.

32. The compound of claim 31 wherein, R⁷ and X are different.

33. The compound of claim 30 wherein,
R⁶ and R⁷ are hydrogen, and
X is ¹⁸F, cyano or CF₃CF₂(CH₂)ₙ—, wherein at least one F is ¹⁸F, and n is zero or one.

34. The compound of claim 30 wherein,
R⁶ and X are hydrogen, and
R⁷ is ¹⁸F, cyano or CF₃CF₂(CH₂)ₙ—, wherein at least one F is ¹⁸F, and n is zero or one.

35. A pharmaceutical composition comprising a compound of claims 1, 21 or 29, and a pharmaceutically acceptable excipient or diluent.

36. A diagnostic composition for imaging serotonin transporters, comprising a compound of claim 1, 21 or 29 and a pharmaceutically acceptable excipient or diluent.

37. A method of imaging serotonin transporters in a mammal, comprising:
(a) introducing into a mammal a detectable quantity of a diagnostic composition of claims 36;
(b) allowing sufficient time for the labeled compound to be associated with serotonin transporters; and
(c) detecting the labeled compound associated with one or more serotonin transporters.

38. A method of following the progression of a therapy which targets SERTs in a mammal, comprising:
(a) introducing into a mammal a detectable quantity of a diagnostic composition of claims 36;
(b) allowing sufficient time for the labeled compound to be associated with serotonin transporters;
(c) detecting the labeled compound associated with one or more serotonin transporters; and
(d) determining an amount of SERTs affected by the therapy.
